(19) United States
(12) Patent Application Publication
(10) Pub. No.: US 2011/0269131 A1
(43) Pub. Date: Nov. 3, 2011

(54) SUBSTRATE FOR MANUFACTURING DISPOSABLE MICROFLUIDIC DEVICES

(76) Inventors: Daniel T. Chiu, Seattle, WA (US); Jason S. Kuo, Seattle, WA (US)

(21) Appl. No.: 13/127,207

(22) PCT Filed: Oct. 28, 2009
(86) PCT No.: PCT/US2009/062426

§ 371 Date: Jul. 20, 2011

Related U.S. Application Data
(60) Provisional application No. 61/109,871, filed on Oct. 30, 2008.

(51) Int. Cl. C12M 1/00; B01D 15/08; C12Q 1/02; G01N 33/559; B01L 3/00; C12Q 1/68

(52) U.S. Cl. 435/6.11; 422/502; 435/6.12; 435/29; 435/283.1; 204/456; 210/635

(57) ABSTRACT

Embodiments of the present invention relate to a UV-curable polyurethane-methacrylate (PUMA) substrate for manufacturing microfluidic devices. PUMA is optically transparent, biocompatible, and has stable surface properties. Embodiments include two production processes that are compatible with the existing methods of rapid prototyping, and characterizations of the resultant PUMA microfluidic devices are presented. Embodiments of the present invention also relate to strategies to improve the production yield of chips manufactured from PUMA resin, especially for microfluidic systems that contain dense and high-aspect-ratio features. Described is a mold-releasing procedure that minimizes motion in the shear plane of the microstructures. Also presented are simple yet scalable able methods for forming seals between PUMA substrates, which avoids excessive compressive force that may crush delicate structures. Two methods for forming interconnects with PUMA microfluidic devices are detailed. These improvements produce a microfiltration device containing closely spaced and high-aspect-ratio fins, suitable for retaining and concentrating cells or beads from a highly diluted suspension.

### CROSS-REFERENCE TO APPLICATION(S) INCORPORATED BY REFERENCE

The present application claims priority to U.S. Provisional Patent Application No. 61/109,871 filed Oct. 30, 2008, entitled “SUBSTRATE FOR MANUFACTURING DISPOSABLE MICROFLUIDIC DEVICES,” and incorporated herein in its entirety by reference.

### TECHNICAL FIELD

The present disclosure is generally directed to devices having enclosed channels and methods for making such devices. More particularly, the present disclosure is directed to microfluidic substrates and microfluidic chips having enclosed channels for accumulating a biological entity.

### BACKGROUND OF THE INVENTION

Microfluidic devices for clinical-diagnostic use have consistently faced a commercialization challenge: how to produce these devices economically such that they can be truly disposable while meeting the material demands of medical use. First-generation microfluidic devices, which were largely developed on silicon or glass substrates, relied heavily on semiconductor processing tools. Because of the heavy capital investment required for processing on these substrates, silicon- or glass-based devices could not be sold inexpensively enough to be disposable.

In the late 1990s, polymer-based rapid prototyping (e.g. molding or embossing) led to a second generation of microfluidic devices. Most notably, polydimethylsiloxane (PDMS) has been a very successful polymeric substrate material for rapid prototyping complex microfluidic systems. Its mix-cast-and-bake method of replication is fast, highly consistent, and simple. As convenient as it is for rapid prototyping, PDMS is not a universal material for all microfluidic applications. Although its elastomeric nature is important for pneumatic valving, this same property makes it prone to expansion when subjected to high fluidic pressure or collapse when high-aspect ratio features or low-aspect ratio channels are involved. Permanent surface modification of PDMS also remains a challenge as its surface has a high tendency to revert back to the hydrophobic state.

Recently, a third wave of microfluidic devices has taken the best of the PDMS replication strategy and addresses some of the shortcomings of PDMS as a substrate for certain types of applications. To increase the production speed, UV-curing instead of thermal curing is increasingly favored. Fiorini, G. S.; Lorenz, R. M.; Kuo, J. S.; Chiu, D. T. Analytical Chemistry 2004, 76, 4697-4704; and Fiorini, G. S.; Yim, M.; Jeffries, G. D. M.; Schiro, P. G.; Mutch, S. A.; Lorenz, R. M.; Chiu, D. T. Lab on a chip 2007, 7, 923-926, explored UV-cured thermoset polyester (TPE) as a complementary substrate material to PDMS. UV-curing of commercial optical adhesives, such as Norland 63, Kim, S. H.; Yang, Y.; Kim, M.; Nam, S. W.; Lee, K. M.; Lee, N. Y.; Kim, Y. S.; Park, S. Advanced Functional Materials 2007, 17, 3494-3498, or custom blends of polyacrylate, Zhou, W. X.; Chan-Park, M. B. Lab on a Chip 2005, 5, 512-518, has been proposed, but invariably due to the choice of resin or photoinitiator, only a thin layer (on the order of 100 μm) can be cured within a reasonable time. To address this issue, Fiorini, et al. used thermal curing after UV exposure to fabricate a microfluidic chip of typical thickness. Additionally, these substrate materials have not been evaluated for medical applications and little is known about resin dissolution, reactivity, solvent residue, or crosslinking byproducts. In particular, no biocompatibility testing has been conducted according to industry guidelines (US Pharmacopeia (USP) or International Organization for Standardization (ISO)), which demonstrates biocompatibility according to an injection test, an intracutaneous test, or an implantation test, on any of the aforementioned materials (PDMS, TPE, Norland optical adhesives, or custom blends of polyacrylate).

As indicated above, PDMS has been an attractive substitute for the fabrication of disposable microfluidic devices; chief among its advantages include the ease of fabrication and its elastomeric nature, which permits facile on-chip valving. However, casting high-aspect-ratio relief features or low-aspect-ratio microchannels is highly challenging in elastomeric PDMS: due to a low shear modulus, frequently microstructures buckle under their own weight, microchannels become pinched off from a sagging ceiling, or apertures expand under increased operating pressure. Efforts to address these mechanical integrity issues include the introduction of harder microfluidic substrates such as h-PDMS (“hard” PDMS), and UV-casting of thermoset polyester (TPE) or commercial optical adhesives, which includes Norland 63 or blends of polyacrylate.

With increasing interest in applying microfluidic devices in clinical applications, it is important to develop substrate materials that are both economical to manufacture and can meet regulatory approval.

### BRIEF SUMMARY OF THE INVENTION

As microfluidic systems transition from research tools to disposable clinical-diagnostic devices, new substrate materials are needed to meet both the regulatory requirement as well as the economics of disposable devices. Embodiments of the present invention introduce a UV-curable polyurethane-methacrylate (PUMA) substrate that has been qualified for medical use and meets all of the challenges of manufacturing microfluidic devices. PUMA is optically transparent, biocompatible, and has stable surface properties. We report two production processes that are compatible with the existing methods of rapid prototyping and present characterizations of the resultant PUMA microfluidic devices.

Particular embodiments of the present invention relate to a new UV-curable polyurethane-methacrylate (PUMA) resin that has excellent qualities as a disposable microfluidic substrate for clinical diagnostic applications. Several strategies are discussed to improve the production yield of chips manufactured from PUMA resin, especially for microfluidic systems that contain dense and high-aspect-ratio features. Specifically, described is a mold-releasing procedure that minimizes motion in the shear plane of the microstructures. Also presented are simple yet scalable methods for forming seals between PUMA substrates, which avoids excessive compressive force that may crush delicate structures. Also detailed are two methods for forming interconnects with PUMA microfluidic devices. These fabrication improvements were deployed to produce a microfiltration device that contained closely spaced and high-aspect-ratio fins, suitable for retaining and concentrating cells or beads from a highly diluted suspension.

### BRIEF DESCRIPTION OF THE DRAWINGS

In order that advantages of the disclosure will be readily understood, a more particular description of aspects of the disclosure briefly described above will be rendered by reference to specific embodiments and the appended drawings. Understanding that these drawings depict only typical embodiments of the disclosure and are not therefore to be considered to be limiting of its scope, the disclosure will be described and explained with additional specificity and detail through the use of the accompanying drawings.

FIGS. 1 and 1′ show procedures for producing a PUMA chip by replicating from a SU-8 master (left branch) and from a silicon master fabricated by deep-reactive-ion-etch (DRIE) (right branch).

FIGS. 2 and 2′ show SEM images of (A) a silanized PDMS imprint and (B) the corresponding PUMA replica. Inset: fine details of the design at a higher magnification.

FIGS. 3 and 3′ show SEM images of various PUMA replica. (A) a 2 μm (H)×4 μm (W) constriction. (B) a two layer channel structure (horizontal channel: 3 μm (W)×3 μm (H); vertical channel: 10 μm (W)×10 μm (H)). (C) A test pattern consisting of solid walls of different widths and regularly spaced columns. (D) Side view of the high-aspect ratio columns shown in (C).

FIGS. 4 and 4′ show (A) Optical transmission characteristics of PUMA, PDMS,

Glass, and TPE. (B) Green fluorescence (solid lines; 510-565 nm, λ_{excitation}=488 nm) and red fluorescence (dashed lines; 660-711 nm, λ_{excitation}=633 nm) intensities of TPE, PUMA, and PDMS. Inset: maximum (initial) autofluorescence of each polymer.

FIGS. 5 and 5′ show PUMA discs submerged for 24 hours in (A) perfluorodecaline, (B) tetrahydrofuran, (C) isopropanol, and (D) 25 μM Rhodamine B (fluorescence image under 533-nm excitation).

FIG. 6 shows electrokinetic characteristics of PUMA substrate. (A) Schematic of the circuit used for EOF measurement. (1: −2 kV Standford PS350 Power Supply; 2: a PUMA chip with a 50 μm (H)×50 μm (W)×3 cm (L) channel filled with borate buffer; 3: 100 kΩ resistor; 4: Keithley 6485 picoammeter; 5: PC for acquiring data). (B) Current traces under electrokinetic-driven flow. Inset: Statistical distribution of v_{eof} measurements; N=68. (C) Current trace as a function of applied electric field. (D) v_{eof} as a function of the age of PUMA chips after bonding.

FIG. 6′ shows electrokinetic characteristics of PUMA substrate. (A) Schematic of the circuit used for EOF measurement. (B) Current traces under electrokinetic-driven flow. Inset: Statistical distribution of v_{eof} measurements; N=68. (C) Current trace as a function of applied electric field. (D) v_{eof} as a function of the age of PUMA chips after bonding.

FIG. 7 shows (A) Layout showing the molding and curing of PUMA chip. A PDMS mold 1 with a recess of 2-mm deep is filled with PUMA resin 2 and embedded with PTFE posts 3. The top of the resin is covered with a clear polypropylene sheet 4 with an interfacial cellophane (or Aclar) sheet 5, which may be peeled off the resin once cured. 1: PDMS mold; 2: PUMA resin; 3: PTFE posts; 4: clear polypropylene sheet; 5: cellophane (or Aclar). (B) Schematic showing two methods to connect external tubings to the chip. Left: PUMA chip 1 with ⅛-in hole can be connected to a barb connector 2 with a ⅛-in OD polyurethane tubing 3; additional PUMA resin 4 may be dispensed around the tubing to prevent leak. Right: PUMA chip 5 with ⅛-in hole can be connected to a 1/16-in OD PTFE tubing 6. 5: PUMA substrate; 6: 1/16-in OD PTFE tubing; 7: polyolefin heat-shrink; 8: retaining ring; 9: additional adhesive; 10: ⅛-in outer-diameter polyurethane tubing; 11: additional PUMA resin.

FIG. 7′ shows (A) Layout showing the molding and curing of PUMA chip. (B) Schematic showing two methods to connect external tubings to the chip.

FIGS. 8 and 8′ show scanning electron microscopy images of (A) PUMA replica of an array of closely spaced high-aspect ratio columns, (B) DRIE-produced silicon master that is opposite in polarity as (A), and (C) PDMS replica made from the silicon master in (B).

FIG. 9 shows a custom-designed release puller for precise release of a PUMA chip from PDMS mold. The Workstation translates downward when the lever is pulled; upon releasing the lever, its spring-loaded action translates upward, ensuring that the PUMA chip is pulled exactly 180 degrees away from the PDMS mold. Gray outline indicates standard Dremel Workstation components 1. A 1-in diameter vinyl suction cup 2 was drilled, mounted, and connected to a vacuum pump via a ⅛-inch (inner diameter) Tygon tubing. A counter-suction cup 3 was mounted below, also connected to vacuum. Metal base 4 was used for securing the counter-suction cup to the Workstation.

FIG. 9′ shows a custom-designed release puller for precise release of a PUMA chip from PDMS mold.

FIGS. 10 and 10′ show (A) defects commonly observed under stereoscope for replication of high-aspect ratio structures. Wavy wall 1 usually results from inadequate cleaning of PDMS mold between each replication run, whereas irregular black spots 2 amidst regular arrays indicate that the structures were leaning against each other (mechanical damage during releasing PUMA from the PDMS mold). (B) SEM image of damaged high-aspect ratio columns; vacuum puller was not used. (C) Optical image of a perfectly released PUMA chip using the vacuum puller described earlier.

FIGS. 11 and 11′ show methods of bonding PUMA chips to form enclosed channels. PUMA chips may be bonded using oxygen plasma first, followed by baking at >75° C. for 23 days. O₂ plasma improves the conformal contact between the chip and the bottom cover. For high-aspect ratio or delicate structures, we recommend the use of a vacuum sealer to control the pressure used in conformal seal. Once good conformal seal is achieved, a permanent bond may be formed by simply subjecting the chip to extended UV exposure, using a programmable infrared oven, or ultrasonic welding.

FIG. 12 shows (A) Retention of MCF-7 cancer cells by high-aspect ratio slits (right side of image) fabricated in PUMA resin. Nominal flow rate was 0.3 ml/min; cells were fixed in 4% paraformaldehyde for 15 min. (B) Retention of 15 μm-diameter beads by high-aspect ratio slits made from PUMA resin. The same microfluidic design was used for (A) and (B), where a filtration barrier comprising the high-aspect ratio slits was placed at the exit of the microchannel.

FIG. 12′ shows (A) Retention or accumulation of MCF-7 cancer cells by high-aspect ratio slits (right side of image) fabricated in PUMA resin. (B) Retention or accumulation of 15 μm-diameter beads by high-aspect ratio slits made from PUMA resin.

FIG. 13 is a cross-sectional view of a microfluidic substrate in accordance with an embodiment of the disclosure.

FIG. 14 is a flow chart illustrating a method for manufacturing a microfluidic substrate using PUMA resin in accordance with an embodiment of the disclosure.

FIGS. 15A-15F are cross-sectional views schematically illustrating stages of a method for manufacturing microfluidic substrates using PUMA resin and by replicating from a SU-8 master in accordance with an embodiment of the disclosure.

FIGS. 16A-16B are cross-sectional views schematically illustrating stages of a method for manufacturing microfluidic substrates using PUMA resin and a silicon master fabricated by deep-reactive-ion-etch in accordance with an embodiment of the disclosure.

### DETAILED DESCRIPTION OF THE INVENTION

Overview

Embodiments of the present disclosure relate to microfluidic substrates and microfluidic chips for accumulating a biological entity. Such substrates may be suitable for use with devices, such as microfluidic devices. In some embodiments, the substrates are formed of a biocompatible material. In other embodiments, the substrate is used to form a microfluidic chip having one or more enclosed flow channels. In further embodiments, the substrate walls absorb radiation.

In one embodiment, a device for accumulating a biological entity is provided. The device can include a flow channel defined at least in part within walls of a biocompatible and radiation absorbing polymer.

Another aspect of the disclosure is directed to a method to form an enclosed microfluidic flow channel. The method can include releasing a formed substrate from a mold. The method can also include providing a vacuum to compress the formed substrate against a surface, and providing an energy to form a seal between the formed substrate and the surface. In one embodiment, the formed substrate is formed by exposing a resin to radiation.

Particular embodiments of the present disclosure relate to a UV-curable polyurethane-methacrylate (PUMA) resin for use as a disposable microfluidic substrate for clinical diagnostic applications. Also disclosed are methods for production of chips manufactured from PUMA resin, especially for microfluidic systems that contain dense and high-aspect-ratio features. For example, one embodiment of a method for producing chips from PUMA resin includes a mold-releasing process that minimizes motion in the shear plane of the microstructures. Also disclosed are simple yet scalable methods for forming seals between PUMA substrates, which can avoid excessive compressive force that can crush delicate structures. Further, two methods for forming interconnects with PUMA microfluidic devices are also disclosed. In another aspect, the present disclosure is directed to a microfiltration device containing closely spaced and high-aspect-ratio fins. In some embodiments, the microfiltration device is suitable for retaining and concentrating cells or beads from a highly diluted suspension.

Further aspects of the disclosure are directed to the use of a device to accumulate a biological entity, wherein the device includes a flow channel defined at least in part within walls of PUMA. In some embodiments, the device can be used for electrophoresis, electrochromatography, high pressure liquid chromatography, filtration, surface selective capture, DNA amplification, polymerase chain reaction, Southern blot analysis, cell culturing, cell proliferation assay, or combinations thereof. In other embodiments, the device can be used for clinical diagnosis.

As used herein, “accumulation” refers to an increase in local density or concentration. Accumulation may occur in a stationary location, in a matrix of materials, or in a mobile phase. Examples of accumulation may include aggregation, concentration, separation, isolation, enriching, focusing, increasing an intensity, or forming sharp bands or spots that can be either stationary or mobile.

Without being limited to the specific examples described herein, “Biological entity” can refer to a cell, an organelle, a subcellular structure, a bacterium, a virus, a protein, an antibody, a DNA or RNA (or aptamer) molecule, an amino acid, a lipid molecule, a bioconjugated particle or other biological or biocompatible material. For example, in one embodiment, the biological entity can be a cell, such as a cancer cell. In some embodiments, the device is suitable for accumulating a biological entity of low-abundance, such as a rare or atypical cell.

Without being limited to the specific examples described herein, a “bioconjugated particle” may include a bioconjugated bead, nanoparticle, magnetic nanoparticle, quantum dot, polymer molecules, or dye molecule.

Embodiments of Substrates for Microfluidic Devices and Microfluidic Devices Including Such Substrates

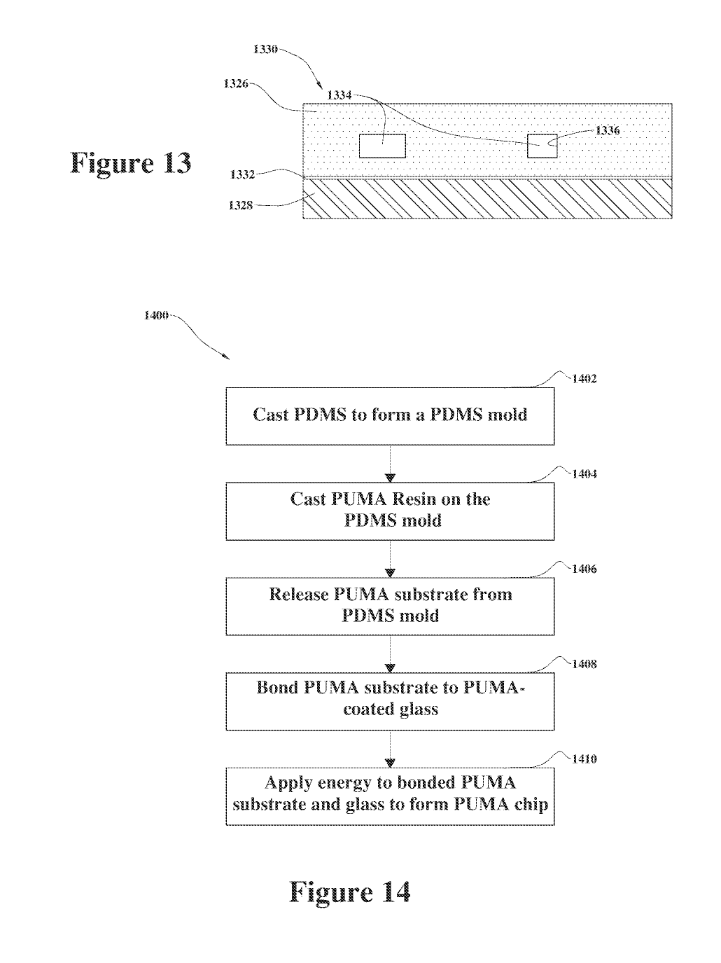

FIG. 13 is a cross-sectional view of a microfluidic chip 1330 in accordance with an embodiment of the disclosure. As shown in FIG. 13, the microfluidic chip 1330 can includes a substrate 1326, such as a PUMA substrate formed from PUMA resin. The microfluidic chip 1330 can also include a glass portion 1328 bonded to the substrate 1326. In one embodiment, the glass portion 1328 is bonded to the substrate 1326 with an adhesive coating layer 1332 on the glass portion 1328. In one embodiment, the adhesive coating layer 1332 includes a medical-grade adhesive such as PUMA. The adhesive coating layer 1332 can be conformally bonded to the substrate 1326, as shown, with applied energy (e.g., Ultraviolet, heat), such that the relief features 1336 are sealed thereby forming one or more flow channels 1334 in the microfluidic chip 1330. In one embodiment, the microfiltration chip 1330 is suitable for retaining and concentrating cells or beads from a highly diluted suspension.

The walls of the flow channel 1334 are constructed from a substrate material possessing certain physical and chemical characteristics. These physical and chemical characteristics include radiation absorption, thermal mechanical response, hardness, elasticity (elastomeric or nonelastomeric), chemical composition, chemical or biological compatibility, surface and interfacial behavior (for example, contact angles or adsorption) and electrical response (for example, generation of electrokinetic flow).

In one embodiment the walls of the substrate 1326 and the relief features 1336 are constructed from a polymer substrate material. In one embodiment the polymer is a thermoplastic. In another embodiment the polymer is nonelastomeric. In a further embodiment the polymer comprises a urethane, an acrylate, a methacrylate, a silicone, or combinations thereof. In one embodiment the microfluidic chip for accumulating a biological entity, such as chip 1330, comprises one or more flow channels 1334 enclosed within walls, such as walls of relief features 1336, that absorb radiation, wherein the walls are formed by cross-linking a medical grade adhesive.

In some embodiments, the substrate 1326 material is a polymer that is biocompatible according to an injection test, an intracutaneous test, or an implantation test, or combinations thereof.

In one embodiment, the polymer, including walls of the relief features 1336, is biocompatible according to an injection test. An injection test may be conducted according to the guidelines for testing medical grade plastics as specified by US Pharmacopeia (USP) or International Organization for Standardization (ISO). As an example, an injection test may be conducted by preparing an extract of said polymer in a sodium chloride solution, a solution of alcohol with sodium chloride, a solution of polyethylene glycol 400, or a vegetable oil, at either 50° C., 70° C., or 121° C., The extracts are then injected into mice. A polymer is deemed biocompatible if none of the animals injected with extracts show reactivity as compared to animals injected with a blank standard.

In another embodiment, the polymer biocompatible according to an intracutaneous test. An intracutaneous test may be conducted according to the guidelines for testing medical grade plastics as specified by US Pharmacopeia (USP) or International Organization for Standardization (ISO). As an example, an intracutaneous test may be conducted by preparing an extract of said polymer in a sodium chloride solution, a solution of alcohol with sodium chloride, a solution of polyethylene glycol 400, or a vegetable oil, at either 50° C., 70° C., or 121° C. The extracts are then injected into rabbits. A polymer is deemed biocompatible if none of the animals injected with extracts show reactivity as compared to animals injected with a blank standard.

In a further embodiment, the polymer is biocompatible according to an implantation test. An implantation test may be conducted according to the guidelines for testing medical grade plastics as specified by US Pharmacopeia (USP) or International Organization for Standardization (ISO). As an example, an implanation test may be conducted by cutting strips of said polymer into not less than 10×1 mm and implanted into rabbits. A polymer is deemed biocompatible if none the implantation sites of polymer strips show reactivity as compared to sites implanted with a control standard.

In some embodiments the walls are constructed from a polymer. In one embodiment the polymer is a thermoplastic. In another embodiment said polymer is nonelastomeric. In another embodiment the polymer comprises a urethane, an acrylate, a methacrylate, a silicone, or combinations thereof. In one embodiment the apparatus for accumulating a biological entity comprises a flow channel enclosed within biocompatible walls that absorb radiation, wherein the walls are formed by crosslinking a medical grade adhesive.

Introduced here is a polyurethane-methacrylate (PUMA) substrate—which has been certified by the supplier as United States Pharmacopeia (USP) Class VI-compliant—as a new material for the manufacturing of microfluidic devices. USP Class VI materials have been tested and proved to be biocompatible and nontoxic according to a systemic injection test, an intracutaneous test, and an implantation test. Along with characterizing the physical, optical, and chemical, and electrokinetic properties of the PUMA microfluidic device, we also report two highly robust replication processes of microstructures and which are compatible with existing replication masters (e.g. SU-8 photoresist on silicon or silicon) so that researchers currently utilizing other rapid-prototyping methods can benefit from using this new substrate.

C. Methods for Manufacturing Microfluidic Substrates

Further aspects of the disclosure are directed to methods for manufacturing substrates described above and devices having such substrates. FIG. 14 is a flow chart illustrating a method 1400 for manufacturing a microfluidic substrate using PUMA resin in accordance with an embodiment of the disclosure. The method 1400 can be used, for example, for replicating fine features onto PUMA substrates. In one embodiment the method 1400 includes casting PDMS to form a PDMS mold (block 1402). In some embodiments, casting PDMS can include casting PDMS on a SU-8 master with relief features to produce a PDMS imprint (i.e., opposite polarity to the relief) with, for example, PDMS channels. In other embodiments, and for replicating high-aspect ratio features, casting PDMS 1402 can include casting a PDMS imprint on a Deep-Reactive Ion Etched (DRIE) silicon master.

The method 1400 also includes casting PUMA resin on the PDMS mold (block 1404) to form a PUMA substrate. The method 1400 further includes releasing the PUMA substrate from the PDMS mold (block 1406). Following step 1406, the method 1400 also includes bonding the PUMA substrate to a PUMA-coated glass substrate (block 1408) and applying ultraviolet and/or heat energy to the bonded PUMA substrate and PUMA-coated glass (block 1410) to form a PUMA chip. In some embodiments, the PUMA chip is a microfluidic substrate suitable, e.g., for use in microfluidic devices such as disposable microfluidic devices.

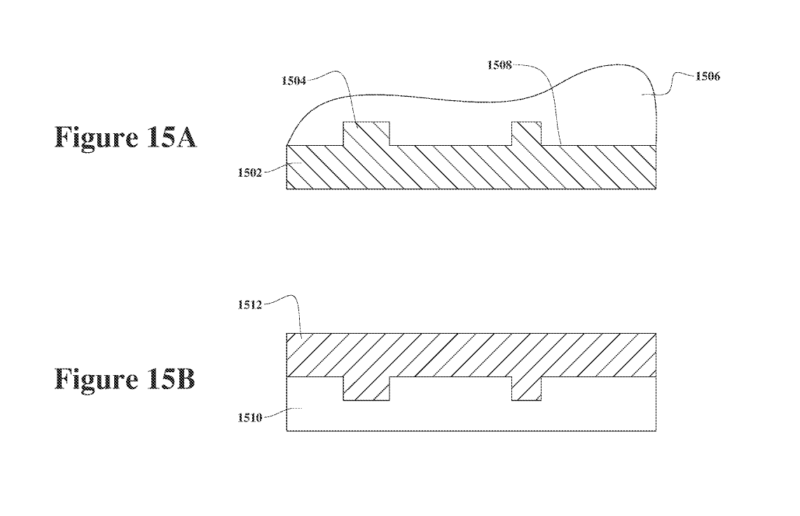

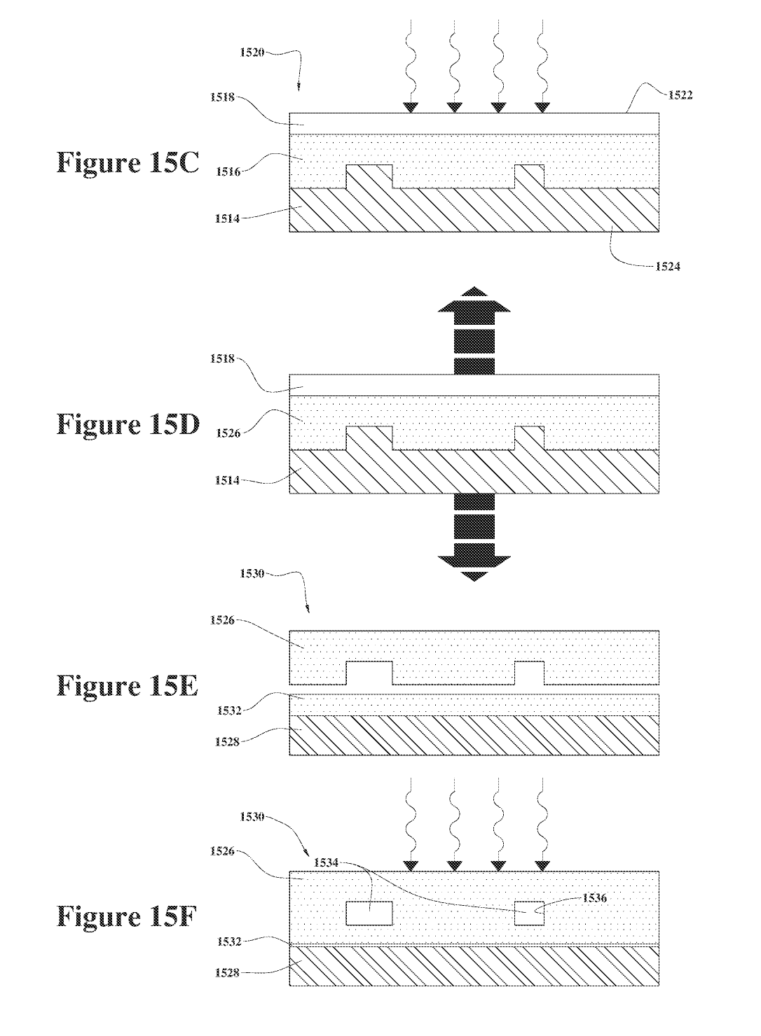

FIGS. 15A-15F are cross-sectional views schematically illustrating stages of a method, such as the method described above with respect to FIG. 14, for manufacturing microfluidic substrates using PUMA resin and by replicating from a SU-8 master in accordance with an embodiment of the disclosure.

FIG. 15A illustrates a SU-8 master 1502 with relief features 1504 used to produce a PDMS imprint (1510; shown in FIG. 15B) having an opposite polarity to the relief features 1504 by pouring (e.g., casting) PDMS material 1506 on to an upper surface 1508 of the SU-8 master 1502. Once the PDMS material is cast, and as shown in FIG. 15B, the PDMS imprint 1510 is oxidized in plasma then silanized with (tridecafluoro-1,1,2,2-tetrahydrooctyl)trichlorosilane in a vacuum dessicator (e.g., to prevent freshly cured PDMS from adhering to the already formed PDMS imprint 1510). A PDMS replica 1512 (i.e., same polarity as the SU-8 master 1502) is produced by pouring additional PDMS on top of the silanized PDMS imprint 1510, curing at 75° C. for at least 2 hr, and separating carefully from the imprint 1510. The PDMS replica 1512 (of the SU-8 master 1502) can then be used as a mold 1514 for PUMA resin 1516 (FIG. 15C). With cleaning between each replication (more details below), the PDMS “master” mold 1514 can be used multiple times. In one embodiment, generating a PDMS replica 1512 of the SU-8 master 1502 can be desirable because PUMA resin 1516 can be difficult to release from a SU-8 master 1502.

FIGS. 15A-15B illustrate steps in the method that utilize existing SU-8 masters used for PDMS replication. However, in another embodiment, the SU-8 master 1502 can be configured with release features 1504 having the same polarity as the desired polarity of the PUMA resin 1516. In this embodiment, the PDMS mold 1514 can be made directly from the Su-8 master without requiring the additional step of making the PDMS imprint 1510.

Referring back to FIG. 15C, PUMA resin 1516 can be dispensed (e.g., at 3-mm thickness) onto the PDMS mold 1514, then covered with a transparent cover 1518, such as a sheet of cellophane tacked to a clear polypropylene backing (e.g., 8-mil thick), to prevent oxygen inhibition of the cross-linking reaction. Aclar sheets (Honeywell, Morristown, N.J.), which is a polychloro-trifluoroethylene (PCTFE) polymer containing no plasticizer, may be used in lieu of cellophane in some applications. To form fluidic reservoirs or holes for external connection, PTFE posts (3 mm (D)×3 mm (H); not shown) can be embedded in the PUMA resin 1516 before curing. The resultant assembly 1520 can be placed in a UV source for 80 sec (expose through PUMA resin side 1522), followed by an additional 40 sec (expose through PDMS mold side 1524) to form a PUMA substrate 1526 (see FIG. 15D). FIG. 15D illustrates a stage in the method wherein the PDMS mold 1514 is removed from the PUMA substrate 1526. Once released from the mold 1514, and as shown in FIG. 15E, PUMA substrate 1526 is conformally bonded to a PUMA-coated (cured) glass 1528 by using gentle mechanical pressure to form a PUMA chip 1530.

As shown in FIG. 15F, a conformal bond between a PUMA coating 1532 on the glass 1528 and the PUMA substrate 1526 is converted to a permanent bond by placing the PUMA chip 1530 under the UV flood source for an additional 10 min. The PUMA chip 1530 can have one or more flow channels 1534 formed between the PUMA substrate 1526 and the PUMA coating 1532. As PUMA material is absorbent to radiation, the walls 1536 of the flow channels 1534 can absorb radiation (e.g., wavelength 300-500 nm).

Between each replication, the PDMS molds 1514 can be sonicated in isopropanol and water and baked at 75° C. for at least 15 min.

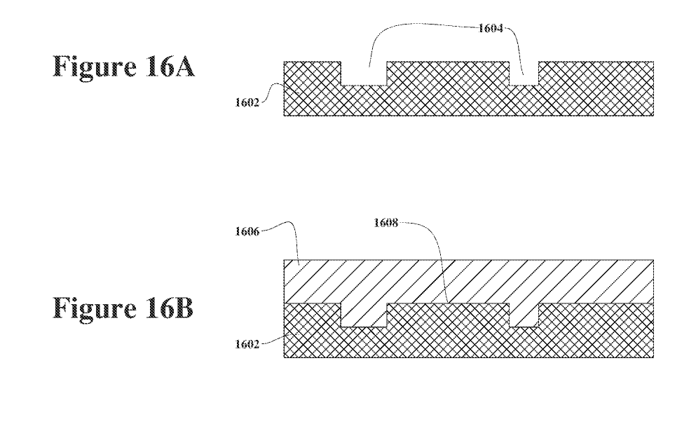

FIGS. 16A-16B are cross-sectional views schematically illustrating stages of a method, such as the method described above with respect to FIG. 13, for manufacturing microfluidic substrates using PUMA resin and a silicon master fabricated by deep-reactive-ion-etch (DRIE) in accordance with an embodiment of the disclosure.

As shown in FIGS. 16A-B, for replicating high-aspect ratio features, a PDMS mold for PUMA casting can be a PDMS imprint casted on a DRIE-Si master. FIG. 16A illustrates a DRIE-Si master 1602 with relief features 1604 used to produce a PDMS mold (such as the PDMS mold 1514 shown in FIG. 15C). As shown in FIG. 16B, by casting PDMS material 1606 on to an upper surface 1608 of the DRIE-Si master 1602, a PDMS mold (such as the PDMS mold 1514 shown in FIG. 15C) having an opposite polarity to the DRIE-Si master 1602 can be formed. The PDMS mold resulting from the steps illustrated in FIGS. 16A-16B can be used to form a PUMA chip as shown in the steps illustrated in FIGS. 15C-15F.

The approach described in FIGS. 16A-16B eliminates the need to produce high-aspect ratio relief features in PDMS, which can be prone to leaning or collapse. Moreover, the approach described in FIGS. 16A-16B can eliminate possible tearing that can occur when separating two inter-digitated pieces of PDMS (e.g., shown in FIG. 15B) such as when the aspect ratio of the microstructure increases.

The disclosure is further illustrated but is not intended to be limited by the following examples.

D. Examples and Additional Embodiments of Substrates, Apparatuses, and Methods of Making and Using such Substrates and Apparatuses

Materials and Methods

Optical Measurement. PUMA substrates (25 mm (W)×75 mm (L)×2 mm (H)) were casted by pouring a UV-curable PUMA resin (140-M Medical/Optical Adhesive, Dymax Corporation) into a PDMS mold. The top surface of the resin was covered with a clear polypropylene sheet (8 mil thickness) with a peelable interfacial sheet of cellophane to prevent oxygen inhibition of the cross-linking reaction. The resin and mold were exposed to a high-intensity UV source (ADAC Cure Zone 2 UV Flood Light Source, fitted with a 400 W metal halide lamp, providing nominally 80 mW/cm² at 365 nm) for 1 min, then flipped over for one additional minute of exposure. The cured PUMA substrate was then released from the mold.

Thermoset polyester (TPE) pieces were prepared as described previously using Polylite 32030-10 resin (Reichhold Company, N.C.).

The optical transmission spectra were collected using a UV-VIS spectrophotometer at 1-nm resolution (Beckman Coulter, DU720). Samples of the TPE, PUMA, and PDMS were all 2-mm thick, but the glass substrate was 1-mm thick. Three spectra were collected for each material and averaged.

Autofluorescence from each material was collected using a custom-built confocal microscope based on a Nikon TE-2000 body. Laser excitation from a solid-state diode pumped 488-nm laser (Coherent Sapphire, Santa Clara, Calif., USA) and a HeNe 633-nm laser was coupled into the back aperture of a 100× objective (N.A. 1.4). Fluorescence was collected by an avalanche photo diode (SPCM-AQR-14, Perkin Elmer, Fremont, Calif., USA). The fluorescence from each material was collected three times in both green wavelength range (510-565 nm) and the red wavelength region (660-710 nm).

Contact-Angle Measurement. PUMA slabs (25 mm (W)×75 mm (L)×3 mm (H)) were prepared using the same protocol as described in the previous section. To compensate for the increased slab thickness, the UV curing time was increased to 80 sec, followed by inverting the PDMS mold and expose through the mold for an additional 40 sec. To determine the effect of plasma oxidation on the surface, three PUMA slabs were subjected to oxygen plasma in a plasma chamber (PDC-001, Harrick Scientific Corp, Ossining, N.Y.) for 6 min (29.6 W applied to the RF coil at a nominal O₂ pressure of 200 mtorr). To characterize the hydrophobic recovery following the plasma oxidation, these oxidized PUMA substrates were sealed in a glass jar and baked in an oven at 75° C. for 2 days.

To measure the contact angle, side profiles of 1-μL MilliQ water droplets on a PUMA substrate were taken with a CCD camera at ambient temperature using the static sessile drop method. Static contact angle between the water-PUMA interface and the water-air interface was measured using the Drop Analysis plug-in in ImageJ software. Contact angle on cured PDMS was also taken for comparison with the literature value. Minimum of triplicate measurements were taken.

Solvent Compatibility. Small PUMA discs were made by casting PUMA resin into a PDMS mold with small circular reservoirs (6 mm (D)×3 mm (H)), covered and cured under UV. The discs were immersed in twenty different chemicals commonly encountered in microfluidic applications for 24 hr at room temperature. Compatibility was determined by observing the change in the circular area of the discs at the end of the experiment. Triplicate samples were collected and the results were averaged. The top image of each disc was captured using a CCD camera under a stereoscope and the circular area was measured using Image) processing software.

Chemicals studied include aqueous or organic solvents, acids, bases, and dyes. To observe the penetration of dyes (Rhodamine B), fluorescence images of the PUMA discs were acquired on a Nikon AZ100 microscope under 533-nm excitation.

Electroosmotic Flow. The microfluidic channel for measuring EOF was a straight channel (50 μm (H)×50 μm (W)×3 cm (L)) with 3-mm (D) fluid reservoirs at the two ends of the channel. The electrical circuit and current-sensing elements follow the current-monitoring method described previously, Huang, X. H.; Gordon, M. J.; Zare, R. N. Analytical Chemistry 1988, 60, 1837-1838; and Locascio, L. E.; Perso, C. E.; Lee, C. S. Journal of Chromotography A 1999, 857, 275-284. A negative-polarity programmable 2 kV DC power supply (Stanford PS350) was connected to a Pt electrode immersed in the cathode reservoir. A second electrode, immersed in the anode reservoir, was connected to a 100 kΩ resistor, in series to a Keithly 6485 picoammeter. The current read by the picoammeter was then recorded by a computer using a custom LabView program, which also controlled the output of the high-voltage power supply. Sodium borate solutions (10 mM and 20 mM) were used as the buffers. The solutions were sonicated immediately prior to use to reduce inadvertent generation of air bubble. PUMA channels were filled by siphoning with a rubber bulb, then the reservoirs were evacuated and refilled with 60 μL of borate solution.

To study the effect of chip age on the electroosmotic mobility, multiple chips were prepared from three separate production runs and then simply stored in petri dishes under ambient conditions. The channels were dry prior to storage, filled with buffer only immediately prior to the EOF measurement. Each chip was used for only one day (i.e., not re-used for EOF measurement on subsequent days).

Results & Discussion

General Physical Properties. The key physical and surface properties of PDMS, TPE, and PUMA are summarized in Table 1.

TABLE 1

| | PDMS | TPE | PUMA |
|---|---|---|---|
| | (Sylgard 184) | (Polylite 32030-10) | (Dymax 140M) |
| Viscosity of Resin | 4600 cp | 450 cp | 3,000 cp¹ |
| After curing | | | |
| Hardness | A50 | 37 | D60 |
| | | (Barcol) | |
| Contact Angle | 120° | 61° | 73° |
| (water-air) | | 42° | 53° |
| | | | 75° |
| Refractive Index | 1.43 | | 1.504 |

PUMA, as based on Dymax 140-M resin, has a comparable viscosity as PDMS (Dow Corning's Sylgard 184), and thus is expected to replicate features as fine as PDMS can. Significantly harder than PDMS, cured PUMA resin is more suitable for producing high-aspect ratio microstructures. Once cured, PUMA is a thermoplastic: although its service temperature as rated by the supplier is between −55 to 200° C., we noticed some softening at >75° C., which can be exploited for bonding. Like PDMS (but unlike TPE), PUMA has very low odor and it is not necessary to handle it under special ventilation.

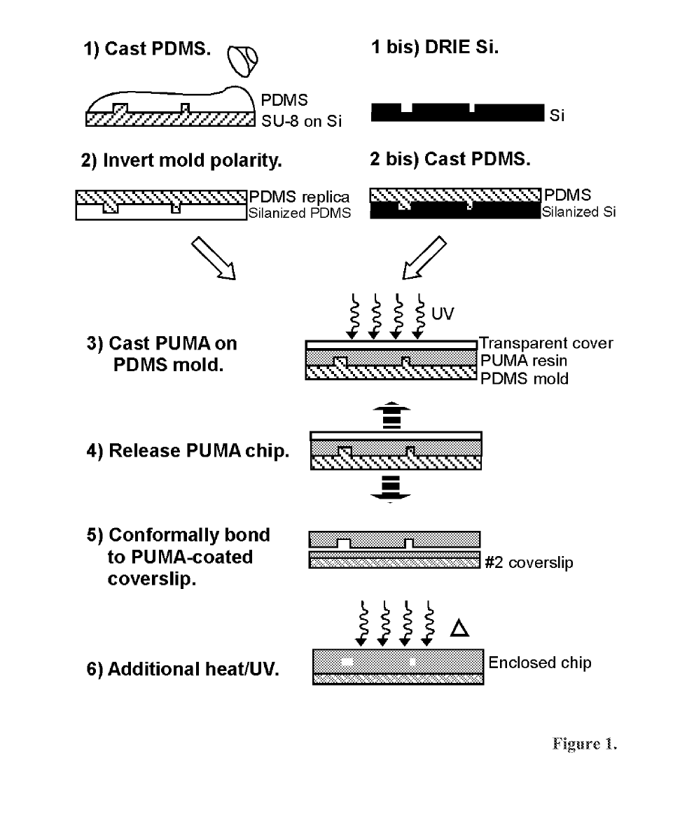

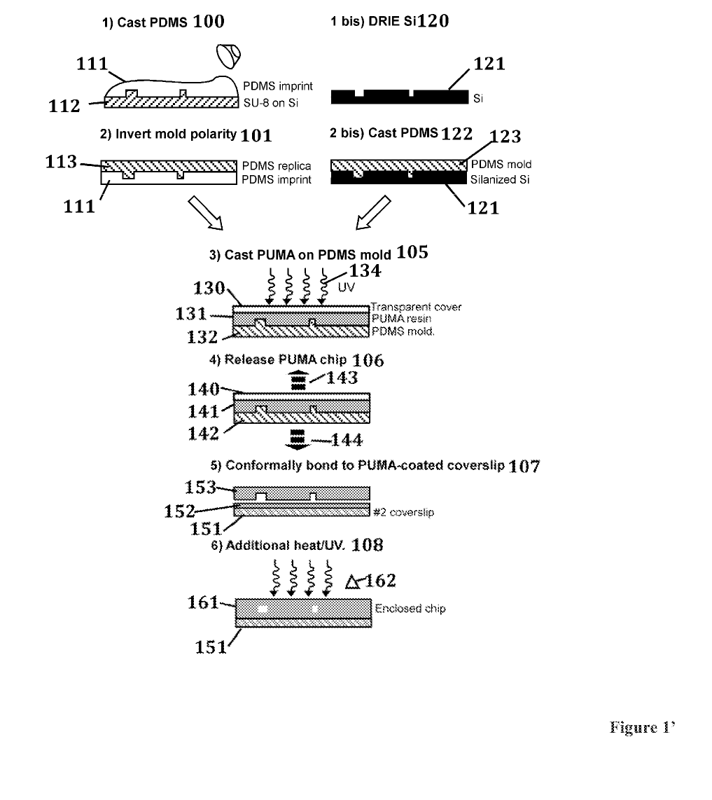

Feature Replication. FIG. 1′ shows a simplified view of a procedures for producing a PUMA chip by replicating from a SU-8 master 112 (left branch) and from a silicon master 121 fabricated by deep-reactive-ion-etch (DRIE) (right branch).

Feature Replication. FIG. 1 shows a simplified view of a procedures for producing a PUMA chip by replicating from a SU-8 master (left branch) and from a silicon master fabricated by deep-reactive-ion-etch (DRIE) (right branch).

FIG. 1′ shows the two procedures used for replicating fine features onto PUMA substrates: the left branch (steps 100, 101, 105, 106, 107, and 108) shows the steps from an SU-8 master 112 that was intended for producing PDMS channels, whereas the right branch (steps 120, 122, 105, 106, 107, and 108) shows the steps from a Deep-Reactive Ion Etched (DRIE) silicon master 121.

FIG. 1 shows the two procedures used for replicating fine features onto PUMA substrates: the left branch shows the steps from an SU-8 master that was intended for producing PDMS channels, whereas the right branch shows the steps from a Deep-Reactive Ion Etched (DRIE) silicon master.

Following the left branch (steps 100, 101, 105, 106, 107, 108) of FIG. 1′, a SU-8 master 112 with relief features was used to produce a PDMS imprint 111 (i.e., opposite polarity to the relief). This PDMS imprint 111 was oxidized in plasma then silanized with (tridecafluoro-1,1,2,2-tetrahydrooctyl)trichlorosilane in a vacuum dessicator; this process prevented freshly cured PDMS from adhering to the already formed PDMS imprint 111. A PDMS replica 113 (i.e., same polarity as the SU-8 master) was produced by pouring additional PDMS on top of the silanized imprint 111, curing at 75° C. for at least 2 hr, and separating carefully from the imprint 111. The PDMS replica 113 (of the SU-8 master) was then used as a mold 132 for PUMA resin 131. With cleaning between each replication (more details below), the PDMS “master” could be used multiple times. This PDMS-on-PDMS replication was needed because PUMA did not release well from SU-8. If the SU-8 master had the correct polarity, then only one PDMS replication would be sufficient. We describe this procedure so that existing SU-8 masters used for PDMS replication can be employed to make a PUMA device.

Following the left branch of FIG. 1, a SU-8 master with relief features was used to produce a PDMS imprint (i.e., opposite polarity to the relief). This PDMS imprint was oxidized in plasma then silanized with (tridecafluoro-1,1,2,2-tetrahydrooctyl)trichlorosilane in a vacuum dessicator; this process prevented freshly cured PDMS from adhering to the already formed PDMS imprint. A PDMS replica (i.e., same polarity as the SU-8 master) was produced by pouring additional PDMS on top of the silanized imprint, curing at 75° C. for at least 2 hr, and separating carefully from the imprint. The PDMS replica (of the SU-8 master) was then used as a mold for PUMA resin. With cleaning between each replication (more details below), the PDMS “master” could be used multiple times. This PDMS-on-PDMS replication was needed because PUMA did not release well from SU-8. If the SU-8 master had the correct polarity, then only one PDMS replication would be sufficient. We describe this procedure so that existing SU-8 masters used for PDMS replication can be employed to make a PUMA device.

After the correct PDMS mold 132 was obtained, PUMA resin 131 was dispensed to 3-mm thickness onto the PDMS mold 132, then covered with a sheet of cellophane tacked to a clear polypropylene backing 130 (8-mil thick) to prevent oxygen inhibition of the cross-linking reaction. Aclar sheets (Honeywell, Morristown, N.J.), which is a polychloro-trifluoroethylene (PCTFE) polymer containing no plasticizer, may be used in lieu of cellophane in critical applications. To form fluidic reservoirs or holes for external connection, PTFE posts (3 mm (D)×3 mm (H)) were embedded in the PUMA resin before curing. The entire assembly was placed in the UV source 134 for 80 sec (expose through resin side), followed by an additional 40 sec (expose through mold). Once released from the mold, PUMA substrate 153 was conformally bonded to another PUMA-coated (cured) glass (152 and 151) by using gentle mechanical pressure and form enclosed channels. This conformal bond was converted to permanent bond by placing the PUMA chip under the UV flood source 162 for an additional 10 min.

After the correct PDMS mold was obtained, PUMA resin was dispensed to 3-mm thickness onto the PDMS mold, then covered with a sheet of cellophane tacked to a clear polypropylene backing (8-mil thick) to prevent oxygen inhibition of the cross-linking reaction. Aclar sheets (Honeywell, Morristown, N.J.), which is a polychloro-trifluoroethylene (PCTFE) polymer containing no plasticizer, may be used in lieu of cellophane in critical applications. To form fluidic reservoirs or holes for external connection, PTFE posts (3 mm (D)×3 mm (H)) were embedded in the PUMA resin before curing. The entire assembly was placed in the UV source for 80 sec (expose through resin side), followed by an additional 40 sec (expose through mold). Once released from the mold, PUMA substrate was conformally bonded to another PUMA-coated (cured) glass by using gentle mechanical pressure. This conformal bond was converted to permanent bond by placing the PUMA chip under the UV flood source for an additional 10 min.

Between each replication, the PDMS molds were sonicated in isopropanol and water and baked at 75° C. for at least 15 min.

For replicating high-aspect ratio features, the mold for PUMA casting was a PDMS imprint 123 casted on a DRIE-Si master 121, as described in the right branch (steps 120, 122, 105, 106, 107, and 108) of FIG. 1′. This approach eliminates the need to produce high-aspect ratio relief features in PDMS, which are prone to leaning or collapse. In addition, two inter-digitated pieces of PDMS, as described in the second step of the left branch (step 101) in FIG. 1′, are highly prone to tear during separation as the aspect ratio of the microstructure increases.

For replicating high-aspect ratio features, the mold for PUMA casting was a PDMS imprint casted on a DRIE-Si master, as described in the right branch of FIG. 1. This approach eliminates the need to produce high-aspect ratio relief features in PDMS, which are prone to leaning or collapse. In addition, two inter-digitated pieces of PDMS, as described in the second step of the left branch in FIG. 1, are highly prone to tear during separation as the aspect ratio of the microstructure increases.

For creating fluidic reservoirs or holes for interconnects, we found embedding PTFE posts to be a simple procedure. Because PUMA is a thermoplastic, laser cutting is also an effective method for creating fluidic reservoirs or interconnect holes. As hole-punching produced significant debris at the walls and caused bending of the substrate at contact points, it is not recommended.

Replication Fidelity. A key challenge in UV casting process is the control of UV dosage according to the thickness of the cast. Because UV light is attenuated as it penetrates the resin, top of the resin is cured first. This results in the top section of the resin becoming over-cured (too stiff) while the interface in contact with the PDMS mold, especially the fine features, remains uncured. To compound the difficulty, the cross-linking reaction of PUMA is moderately inhibited by PDMS. Although elastomeric silicones have excellent release properties, excessive UV curing did lead to permanent bonding between the resin and the mold. Thus a window of time exists for the optimal UV exposure and the exposure must be done both from above the resin as well as through the transparent mold. This window must be individually mapped out for each UV exposure source. In the event the window of time is too short to be precisely followed by manual operation, more tolerance may be granted by decreasing the photon flux, for example, by either using a lower intensity light source or placing plates of glass above the resin to attenuate the intensity.

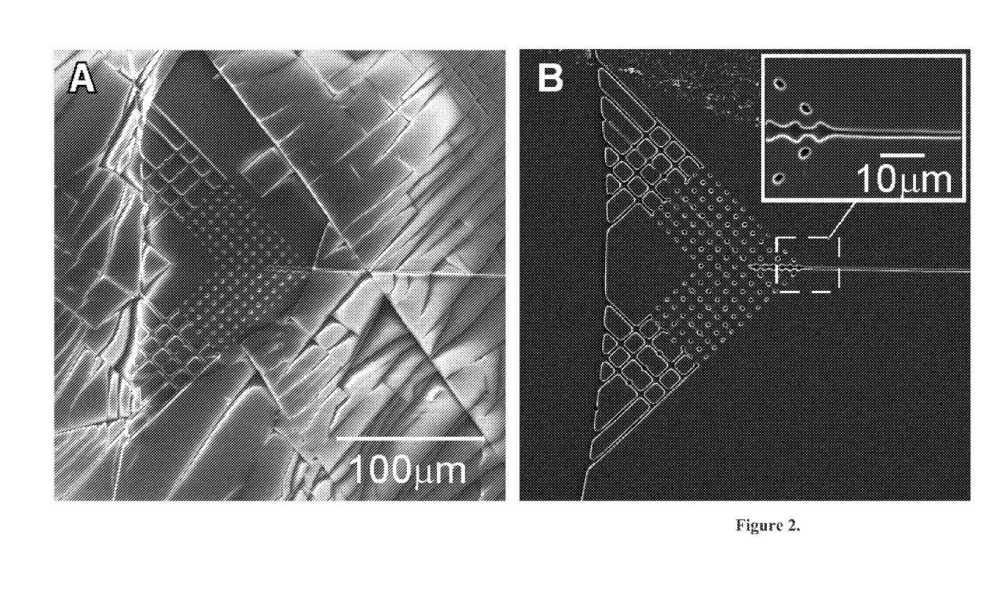

FIG. 2′ shows SEM images of (A) a silanized PDMS imprint 210 and (B) the corresponding PUMA replica 220. The inset 230 shows fine details of the design at a higher magnification. FIG. 2′A shows the SEM image of a silanized PDMS imprint 210 and FIG. 2′B shows the corresponding PUMA replica 220 (same polarity as the imprint).

FIG. 2 shows SEM images of (A) a silanized PDMS imprint and (B) the corresponding PUMA replica. The inset shows fine details of the design at a higher magnification. FIG. 2A shows the SEM image of a silanized PDMS imprint and FIG. 2B shows the corresponding PUMA replica (same polarity as the imprint).

This PUMA replica 220 was produced using the two-step PDMS transfer method described according to the left branch of FIG. 1′ (steps 100, 101, 105, 106, 107, and 108). The replication fidelity was excellent, down to ˜2 μm as shown in the inset 230 of FIG. 2′B. We note that the SEM image of PDMS imprint 210 exhibited significant surface cracking 211; these cracks 211 were long enough to be visible to naked eyes but they appeared to be very fine and superficial. We have consistently observed this surface cracking behavior in the SEM images of PDMS that have been subjected to plasma bombardment, either from oxygen plasma treatment or sputtering of Au/Pd thin coating during SEM sample preparation. For most cases these surface cracks were not seen in the PUMA replica 220.

This PUMA replica was produced using the two-step PDMS transfer method described according to the left branch of FIG. 1. The replication fidelity was excellent, down to ˜2 μm as shown in the inset of FIG. 2B. We note that the SEM image of PDMS imprint exhibited significant surface cracking; these cracks were long enough to be visible to naked eyes but they appeared to be very fine and superficial. We have consistently observed this surface cracking behavior in the SEM images of PDMS that have been subjected to plasma bombardment, either from oxygen plasma treatment or sputtering of Au/Pd thin coating during SEM sample preparation. For most cases these surface cracks were not seen in the PUMA replica.

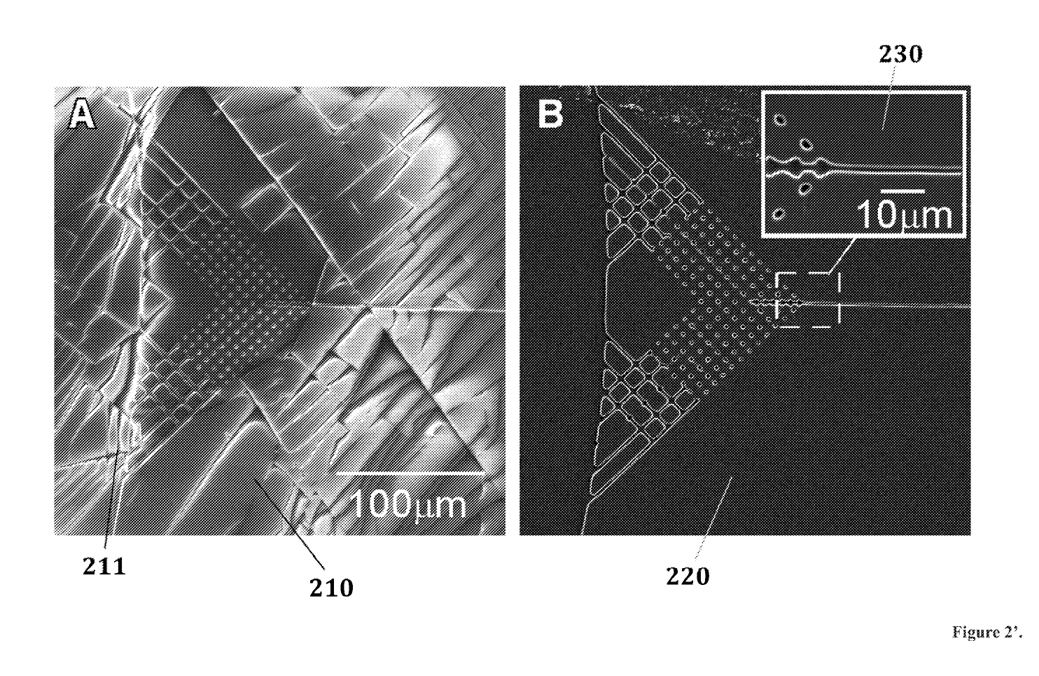

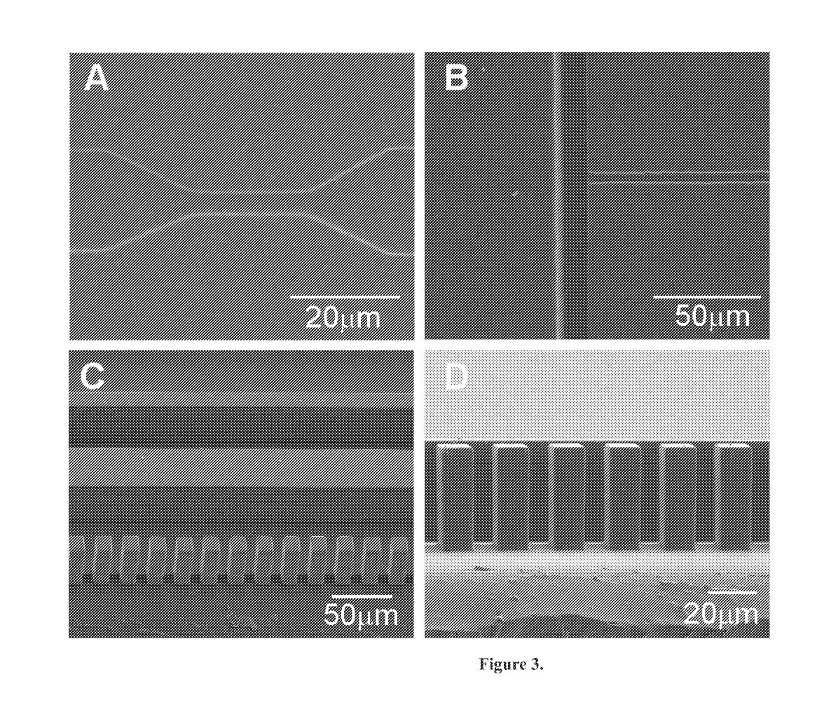

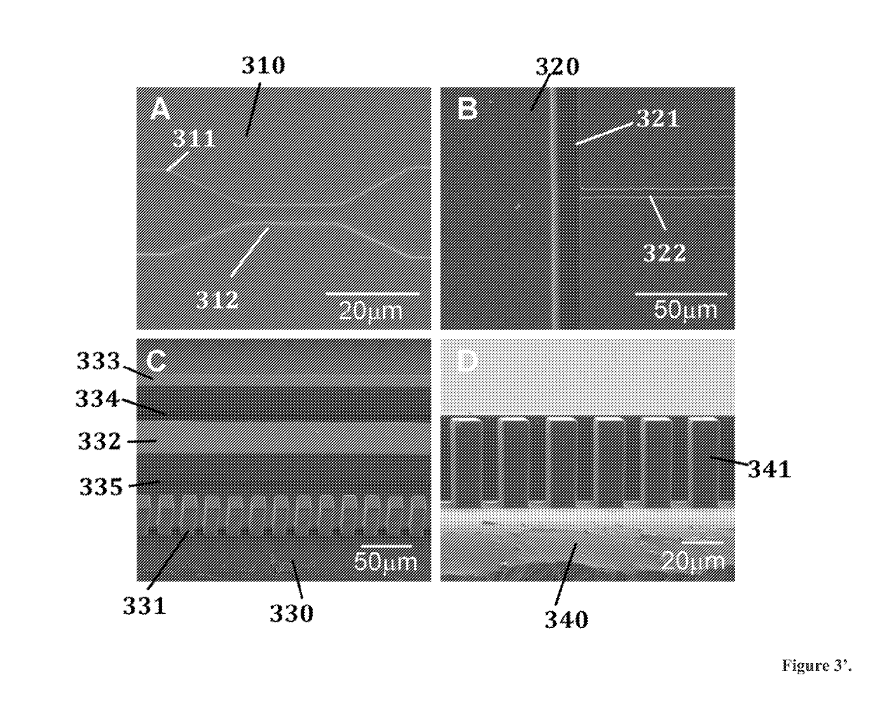

FIG. 3′ shows SEM images of various PUMA replicas 310, 320, 330, 340. FIG. 3′(A) shows a 2 μm (H)×4 μm (W) constriction 312. FIG. 3′(B) a two layer channel structure (horizontal channel 322: 3 μm (W)×3 μm (H); vertical channel 321: 10 μm (W)×10 μm (H)). FIG. 3′(C) shows a test pattern consisting of solid walls (332, 333) of different widths and regularly spaced columns 331. FIG. 3′(D) shows a side view of the high-aspect ratio columns 331 shown in (C).

FIG. 3 shows SEM images of various PUMA replicas. FIG. 3(A) shows a 2 μm (H)×4 μm (W) constriction. FIG. 3(B) a two layer channel structure (horizontal channel: 3 μm (W)×3 μm (H); vertical channel: 10 μm (W)×10 μm (H)). FIG. 3(C) shows a test pattern consisting of solid walls of different widths and regularly spaced columns. FIG. 3(D) shows a side view of the high-aspect ratio columns shown in (C).

In particular, FIG. 3′ shows more SEM images of microstructures replicated into PUMA. FIG. 3′A shows a PUMA replica 310 of a 2-μm tall microchannel constriction 312 that is 4-μm wide at the neck. As can be seen in the SEM image, the details of the channel tapering 311 were well preserved. FIG. 3′B is a two-layer structure: the two orthogonal channels 321 and 322 were of different height; the horizontal channel 322 was 3 μm (W)×3 μm (H), whereas the vertical channel 321 was 10 μm (W)×10 μm (H). Two-layer structure did not pose any problem for the mold-releasing step.

In particular, FIG. 3 shows more SEM images of microstructures replicated into PUMA. FIG. 3A shows a PUMA replica of a 2-μm tall microchannel constriction that is 4-μm wide at the neck. As can be seen in the SEM image, the details of the channel tapering were well preserved. FIG. 3B is a two-layer structure: the two orthogonal channels were of different height; the horizontal channel was 3 μm (W)×3 μm (H), whereas the vertical channel was 10 μm (W)×10 μm (H). Two-layer structure did not pose any problem for the mold-releasing step.

FIG. 3′C shows the SEM image of a test pattern consisting of alternating solid walls (332 and 333) of various width and spacing (334 and 335) replicated in PUMA. Unlike the replicas (310 and 320) shown in FIG. 3′A and 3′B, the replica 330 in FIG. 3′C was obtained by following the right branch of the procedure (steps 120, 122, 105, 106, 107, and 108) outlined in FIG. 1′; in other words, the replication process originated from a DRIE-etched Si master 121. This test pattern was developed to test if (1) UV crosslinking may have been non-uniform as a function of feature density, and (2) dense features may have been more prone to damage from mold releasing. The height of the microstructures was ˜40 μm. FIG. 3′D is a profile-view of the columns 331 in the lower half of FIG. 3′C: these densely-spaced columns 341 had sharp, crisp sidewalls with no evidence of leaning or broadening. The aspect ratio (H/W) achieved in this case was ˜3.5.

FIG. 3C shows the SEM image of a test pattern consisting of alternating solid walls of various width and spacing replicated in PUMA. Unlike the replicas shown in FIGS. 3A and 3B, the replica in FIG. 3C was obtained by following the right branch of the procedure outlined in FIG. 1; in other words, the replication process originated from a DRIE-etched Si master. This test pattern was developed to test if (1) UV crosslinking may have been non-uniform as a function of feature density, and (2) dense features may have been more prone to damage from mold releasing. The height of the microstructures was ˜40 μm. FIG. 3D is a profile-view of the columns in the lower half of FIG. 3C: these densely-spaced columns had sharp, crisp sidewalls with no evidence of leaning or broadening. The aspect ratio (H/W) achieved in this case was ˜3.5.

Contact Angle. For comparison with the literature value, the contact angle of water on native PDMS as measured on our setup was 102°, which is consistent with that reported by Hillborg, et al. The UV-cured PUMA substrate had a contact angle of 72°, which is significantly more hydrophilic compared to PDMS. This value is very close to the reported value of polyurethane, which is a major component of this resin. Treatment with oxygen plasma further reduced the contact angle of PUMA to 53°, which is also in agreement with that of oxidized polyurethane. Plasma reduction of contact angle was reversed by baking; the contact angle returned to 75°, which is within statistical agreement with the native PUMA substrate.

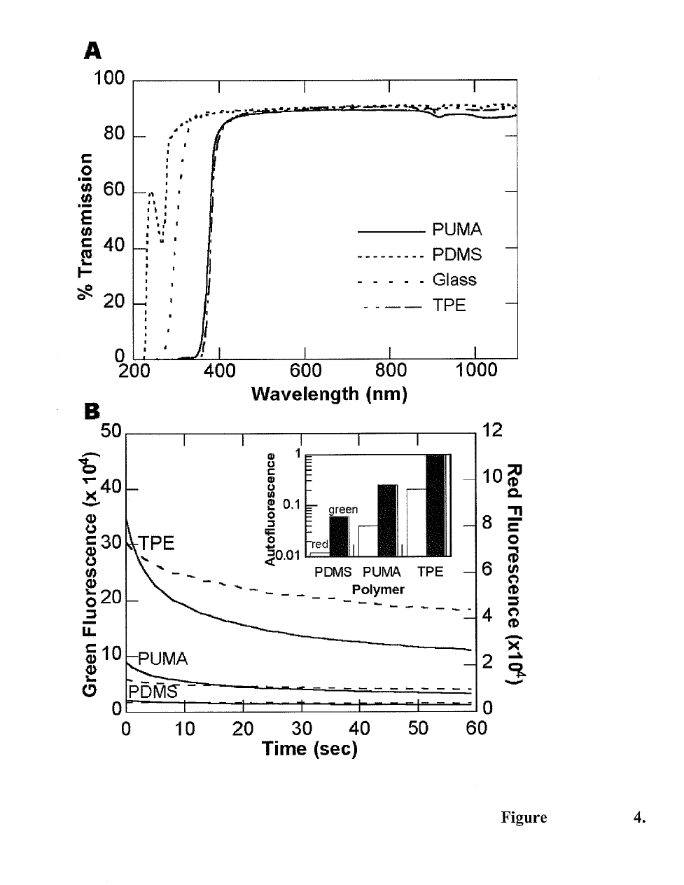

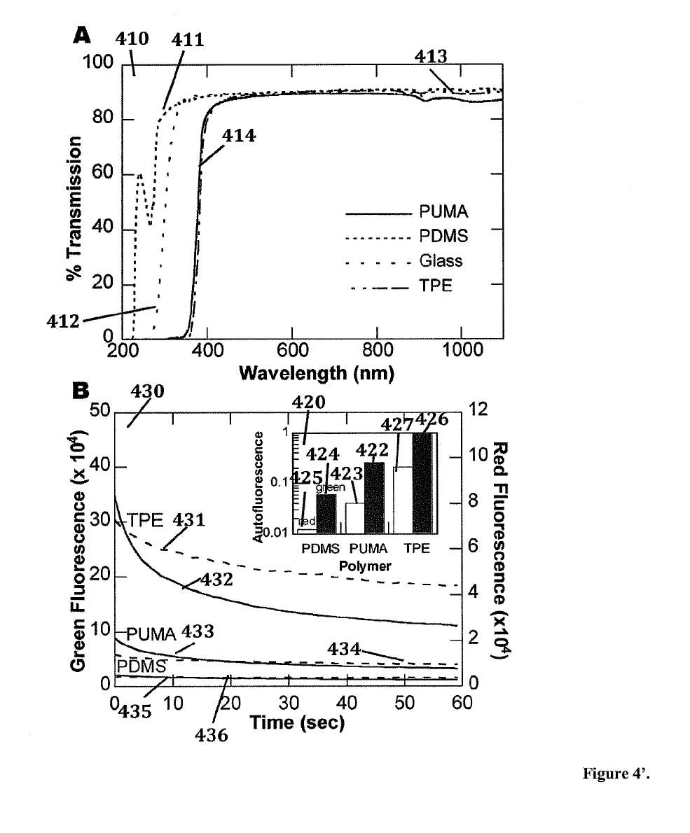

Optical Properties. Cured PUMA is optically clear, with a refractive index of 1.504. FIG. 4′A shows optical transmission characteristics 410 of PUMA 414, PDMS 411, Glass 412, and TPE 413. FIG. 4′B shows green fluorescence (solid lines 432, 433, 435; 510-565 nm, λ_{excitation}=488 nm) and red fluorescence (dashed lines 431, 434, 436; 660-711 nm, λ_{excitation}=633 nm) intensities of TPE, PUMA, and PDMS. Inset: maximum (initial) autofluorescence of each polymer.

Optical Properties. Cured PUMA is optically clear, with a refractive index of 1.504. FIG. 4(A) shows optical transmission characteristics of PUMA, PDMS, Glass, and TPE. FIG. 4(B) shows green fluorescence (solid lines; 510-565 nm, λ_{excitation}=488 nm) and red fluorescence (dashed lines; 660-711 nm, λ_{excitation}=633 nm) intensities of TPE, PUMA, and PDMS. Inset: maximum (initial) autofluorescence of each polymer.

In one embodiment, the device for accumulating a biological entity comprises a flow channel defined at least in part within walls of a biocompatible and radiation-absorbing polymer. In another embodiment, the device for accumulating a biological entity comprises a flow channel defined at least in part within walls of a biocompatible and radiation-absorbing polymer, wherein the polymer comprises polyurethane-methacrylate (PUMA). In a further embodiment, the device for accumulating a biological entity comprises a flow channel defined at least in part within walls of a biocompatible and radiation-absorbing polymer, wherein the polymer comprises a urethane, an acrylate, a methacrylate, a silicone, or combinations thereof.

In one embodiment, the device for accumulating a biological entity comprises a flow channel defined at least in part within walls of a biocompatible and radiation-absorbing polymer, wherein the polymer is biocompatible according to an injection test, an intracutaneous test, an implantation test, or combinations thereof.

In one embodiment, the device for accumulating a biological entity comprises a flow channel defined at least in part within walls of a biocompatible and radiation-absorbing polymer, wherein the walls are formed by crosslinking a medical grade adhesive.

In one embodiment, a device for accumulating a biological entity comprises a flow channel defined at least in part within walls of a biocompatible and radiation-absorbing polymer, wherein the the polymer absorbs radiation at wavelengths between 300-500 nm. In another embodiment, the device for accumulating a biological entity comprises a flow channel defined at least in part within walls of a biocompatible and radiation-absorbing polymer, wherein the polymer absorbs radiation at wavelengths between 350-500 nm.

In one embodiment, the device for accumulating a biological entity comprises a flow channel defined at least in part within walls of a biocompatible and radiation-absorbing polymer, wherein the polymer absorbs more than 20% radiation at wavelengths between 300-500 nm, or in another embodiment, between 350-500 nm. As shown in trace 412 of FIG. 4′A, PDMS transmits more than 80% and does not absorb more than 20% radiation between 300-500 nm.

In a further embodiment, the device for accumulating a biological entity comprises a flow channel defined at least in part within walls of a biocompatible and radiation-absorbing polymer, wherein the polymer absorbs less than 20% radiation at wavelengths between 500-1000 nm but more than 20% between 350-500 nm. The optical transmission of walls manufactured from PUMA resin as shown in FIG. 4′A indicates optical transparency (>80% transmission) in the visible spectrum range (500-1000 nm), and rapidly became opaque (no transmission) in the UV range (350-500 nm) as the radiation was absorbed by the resin.

FIG. 4′A plots the optical transmission through PUMA, from which the channel walls are constructed, over 200-1000 nm wavelength. The optical transmission dropped precipitously in the range of 300-500 nm, indicating a strong absorbance of UV radiation.

FIG. 4′A plots the optical transmission through PUMA (trace 414) over 200-1000 nm, along with that of TPE (trace 413), PDMS (trace 411), and glass (trace 412). PUMA has a similar optical clarity as glass in the visible range; however, because of the strong residual presence of UV photoinitiator for crosslinking, one expects a sharp absorption in the UV range.

FIG. 4A plots the optical transmission through PUMA over 200-1000 nm, along with that of TPE, PDMS, and glass. PUMA has a similar optical clarity as glass in the visible range; however, because of the presence of UV photoinitiator for crosslinking, one naturally expects a sharp absorption in the UV range. Thus PUMA, like TPE, is not particularly suitable for UV absorbance applications.

In one embodiment, the device for accumulating a biological entity comprises a flow channel defined at least in part within walls of a biocompatible and radiation-absorbing polymer, wherein the walls do not autofluorescence. For example, in some embodiments, the walls exhibit no autofluorescence under 488-nm illumination. In other embodiments, the walls exhibit no autofluorescence under 633-nm illumination.

FIG. 4′B shows the autofluorescence by the polymer substrates under 488- and 633-nm excitation. The autofluorescence level (431, 432, 433, 434, 435, 436) of all three polymer substrates decayed over time, consistent with observations in other plastic materials. FIG. 4′B inset compares the maximum autofluorescence level of PDMS (424, 425), PUMA (422, 423), and TPE (426, 427): PUMA exhibited less autofluorescence than TPE but more than PDMS. This level of autofluorescence is suitable for most applications involving fluorescence detection. For high-sensitivity single-molecule work, however, a confocal detection geometry that can efficiently reject background signal from the substrate can be employed.

FIG. 4B shows the autofluorescence by the polymer substrates under 488- and 633-nm excitation. The autofluorescence level of all three polymer substrates decayed over time, consistent with observations in other plastic materials. FIG. 4B inset compares the maximum autofluorescence level of PDMS, PUMA, and TPE: PUMA exhibited less autofluorescence than TPE but more than PDMS. This level of autofluorescence is suitable for most applications involving fluorescence detection. For high-sensitivity single-molecule work, however, a confocal detection geometry that can reject efficiently background signal from the substrate should be employed.

In one embodiment, the device for accumulating a biological entity comprises a flow channel defined at least in part within walls of a biocompatible and radiation-absorbing polymer, wherein the walls are resistant against an oil, an acid or a base. For example, the walls can be resistant against mineral oil, Fluorinert oil, perfluorodecaline, or silicone oil.

Solvent Compatibility. Table 2 tabulates the observed swelling ratio of PUMA discs in each chemical.

TABLE 2

| Chemical | Area Ratio PUMA |
|---|---|
| Acetic acid, 1M | 1.0 |
| Hydrochloric acid, 1M | 1.0 |
| Ammonium Hydroxide, 1M | 1.0 |
| Sodium Hydroxide, 1M | 1.0 |
| Acetone | 1.3 |
| Acetonitrile | 1.1 |
| DMSO | 1.5 |
| Formaldehyde | 1.0 |
| Heptane | 1.1 |
| Tetrahydrofuran | 1.8 |
| Methanol | 1.4 |
| Ethanol | 1.4 |
| 2-Propanol | 1.2 |
| Fluorescein | 1.0 |
| Rhodamine B | 1.0 |
| Fluorinert | 1.0 |
| Mineral oil | 1.0 |
| Perfluorodecalin | 1.0 |
| Silicone oil | 1.0 |
| Water | 1.0 |

PUMA was found to be very resistant to dyes, acids, bases, water, formaldehyde, mineral oil, silicone oil, Fluorinert, and perfluorodecaline. While most organic solvents at 100% purity caused swelling, PUMA had lower swelling ratios with acetone and acetonitrile than those of TPE. We note that for low molecular weight alcohols such as methanol and ethanol, PUMA appears to have swollen more comparing to polyurethane alone, which had a swelling ratio of ˜1.1.

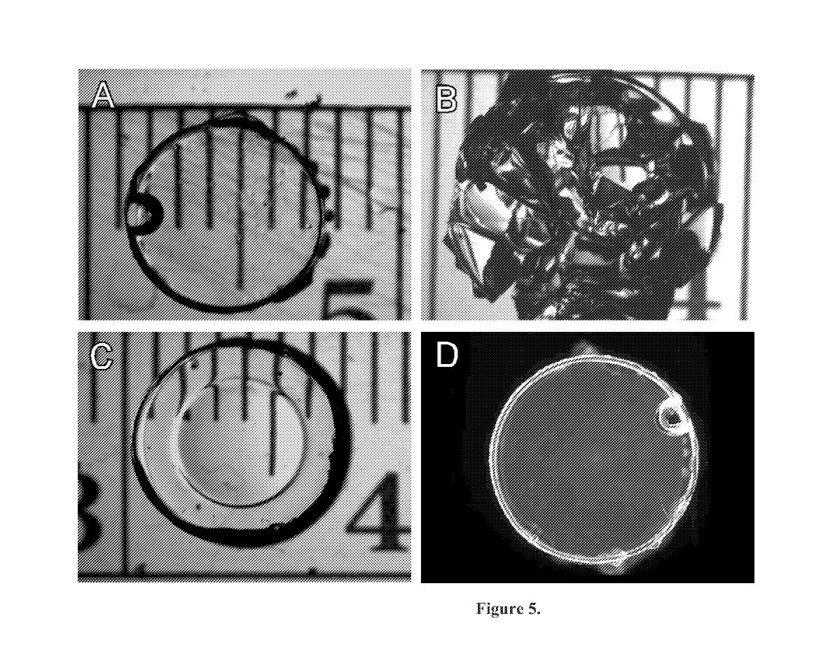

FIG. 5′ shows PUMA discs 510, 520, 530, and 540 submerged for 24 hours in (A) perfluorodecaline, (B) tetrahydrofuran, (C) isopropanol, and (D) 25 μM Rhodamine B (fluorescence image under 533-nm excitation). FIG. 5′ shows select images of PUMA discs 510, 520, 530, and 540 after immersion for 24 hr in various organic compounds and dyes to illustrate the effects of immersion. Oils immiscible with water had no effect on the PUMA discs 510 (FIG. 5′A). We also conducted additional testing of PUMA by heating samples in mineral oil, Fluorinert, and perfluorodecaline up to 90° C.; no apparent change in circular area or dissolution was observed. Accordingly, PUMA can be compatible with emerging applications in droplet microfluidics, which employ many of these oils. On the other hand, significant swelling was observed in the alcohols, heptane, DMSO, and in particular, tetrahydrofuran, in which severe cracking was observed (FIG. 5′B, disc 520). For some solvents, rather than causing a uniform expansion, some discs 530 formed a depression 532 in the center as a result of immersion (FIG. 5′C, with isopropanol). This is likely due to a slower rate of penetration such that after 24 hr the center of the disc remained largely unaffected.

FIG. 5 shows PUMA discs submerged for 24 hours in (A) perfluorodecaline, (B) tetrahydrofuran, (C) isopropanol, and (D) 25 μM Rhodamine B (fluorescence image under 533-nm excitation). FIG. 5 shows select images of PUMA discs after immersion for 24 hr in various organic compounds and dyes to illustrate the effects of immersion. Oils immiscible with water had no effect on the PUMA discs (FIG. 5A). We also conducted additional testing of PUMA by heating samples in mineral oil, Fluorinert, and perfluorodecaline up to 90° C.; no apparent change in circular area or dissolution was observed. This fact should make PUMA compatible with emerging applications in droplet microfluidics, which employ many of these oils. On the other hand, significant swelling was observed in the alcohols, heptane, DMSO, and in particular, tetrahydrofuran, in which severe cracking was observed (FIG. 5B). For some solvents, rather than causing a uniform expansion, some discs formed a depression in the center as a result of immersion (FIG. 5C, with isopropanol). This is likely due to a slower rate of penetration such that after 24 hr the center of the disc remained largely unaffected.

Dye penetration was observed in PUMA discs 540 immersed in 25 μM Rhodamine B (FIG. 5′D) but was not observed in fluorescein. Dye penetration by Rhodamine B is disappointing but not unexpected as Rhodamine B is known to penetrate most polymeric materials.

Dye penetration was observed in PUMA discs immersed in 25 μM Rhodamine B (FIG. 5D) but was not observed in fluorescein. Dye penetration by Rhodamine B is disappointing but not unexpected as Rhodamine B is known to penetrate most polymeric materials.

In one embodiment the device for accumulating a biological entity comprises a flow channel defined at least in part within walls of a biocompatible and radiation-absorbing polymer, wherein the flow channel generates an electrokinetic flow.

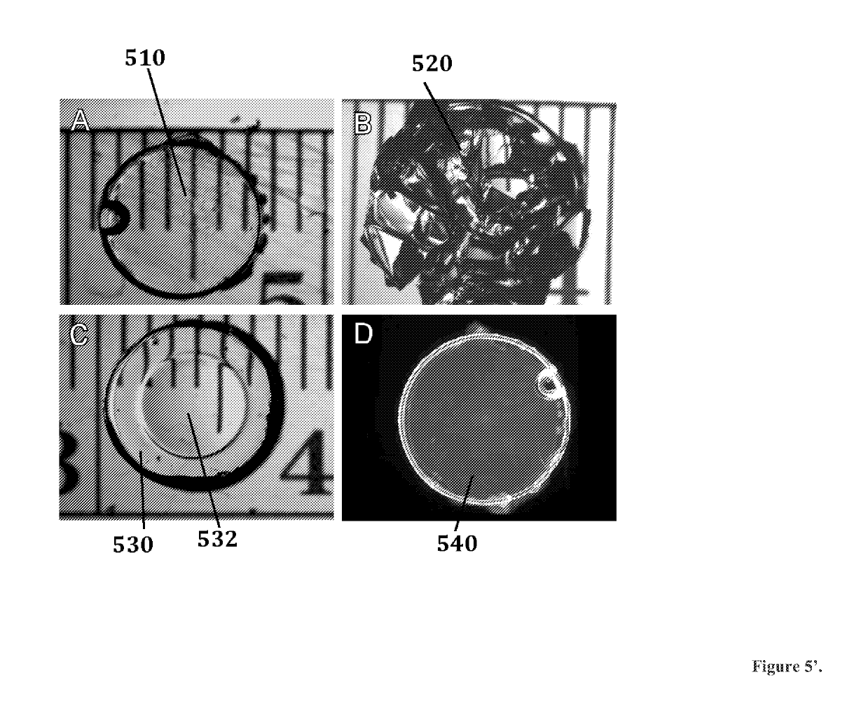

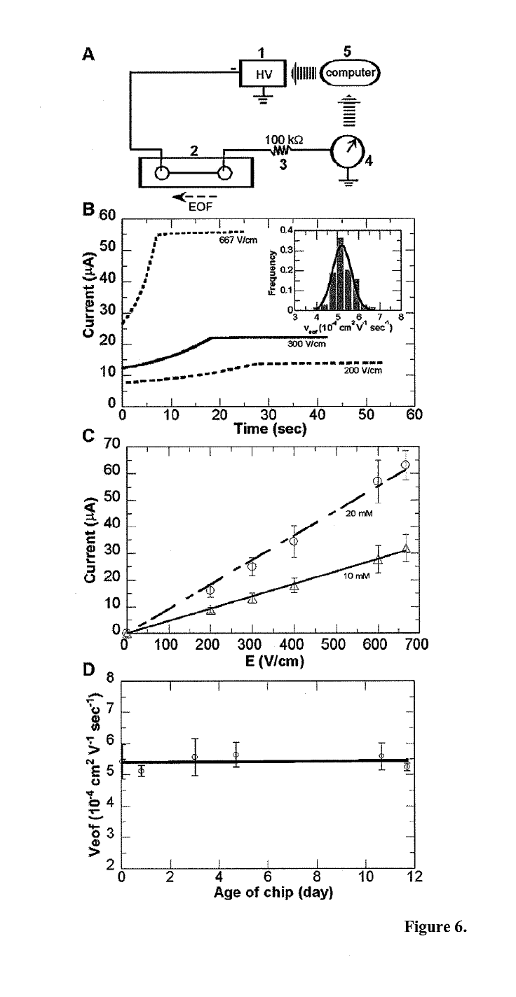

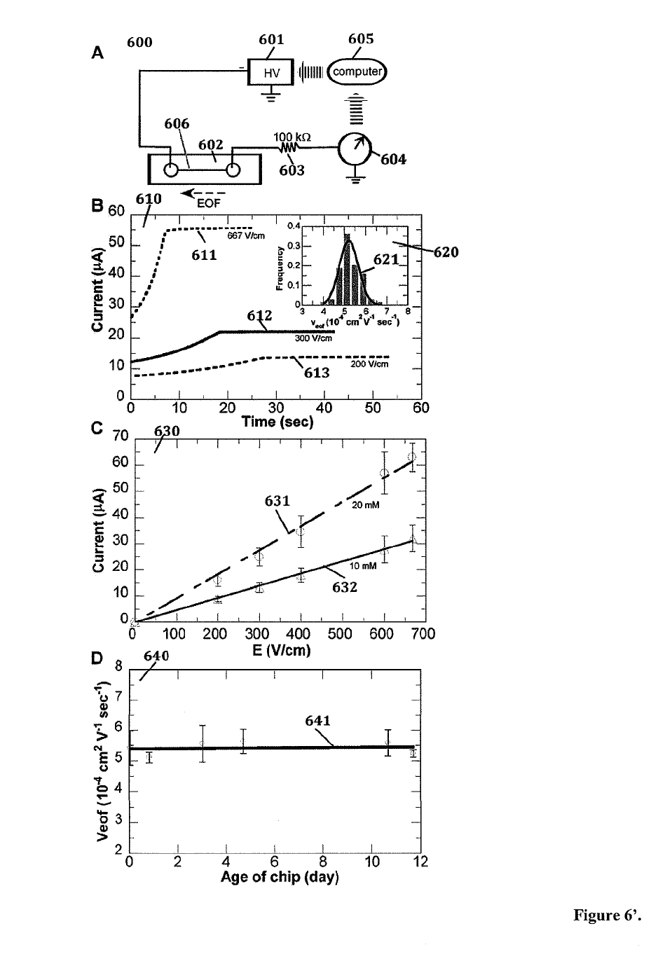

Electroosmotic Flow. FIG. 6′A shows the electrical circuit of the EOF experiment. FIG. 6′ shows electrokinetic characteristics of PUMA substrate. FIG. 6′A is a schematic of the circuit used for EOF measurement. (601: −2 kV Standford PS350 Power Supply; 602: a PUMA chip with a 50 μm (H)×50 μm (W)×3 cm (L) channel 606 filled with borate buffer; 603: 100 kΩ resistor; 604: Keithley 6485 picoammeter; 605: PC for acquiring data). FIG. 6′B shows current traces 611, 612, and 613 under electrokinetic-driven flow. The inset 620 shows statistical distribution of v_{eof} measurements; N=68. FIG. 6′C shows current traces 631 and 632 as a function of applied electric field. FIG. 6′D plots v_{eof} (641) as a function of the age of PUMA chips after bonding. Native PUMA exhibited very strong electroosmotic mobility; the EOF moves toward cathode, the same direction as in PDMS, glass, and TPE. This would suggest that the native PUMA surface also exhibited negative charge under the buffer environment used. In borate buffer, v_{eof}, the electroosmotic mobility of PUMA, was 5.5×10⁻⁴ cm²V⁻¹sec⁻¹, quite comparable to that of fused-silica capillary; FIG. 6′B inset (620) shows the statistical distribution of electroosmotic mobility measurements. This value is ˜2 times higher than that of thermal-cured polyurethane reported in the literature. FIG. 6′B shows how the electrical current 611, 612, and 613 stabilized when the anode reservoir was replaced with 20-mM borate buffer. As the EOF drove the 20-mM buffer solution in anode reservoir to displace the 10-mM buffer previously in the channel, the ionic strength increased and led to an increase of electrical current until the entire channel was filled with 20-mM buffer. As the electric field increased from 200 V/cm to 667 V/cm (the maximum output from our power supply), the time to reach a new steady state decreased as expected. Within the range of electric field that we applied, we did not notice any Joule heating. FIG. 6′C plots the electrical current 631 and 632 measured using 10- and 20-mM borate buffers as a function of the applied electric field. Up to 667 V/cm, these relationships were linear, indicating no alteration in ionic conductivity from Joule heating.

Electroosmotic Flow. FIG. 6A shows the electrical circuit of the EOF experiment. FIG. 6 shows electrokinetic characteristics of PUMA substrate. FIG. 6(A) is a schematic of the circuit used for EOF measurement. (1: −2 kV Standford PS350 Power Supply; 2: a PUMA chip with a 50 μm (H)×50 μm (W)×3 cn (L) channel filled with borate buffer; 3: 100 kΩ resistor; 4: Keithley 6485 picoammeter; 5: PC for acquiring data). FIG. 6(B) shows current traces under electrokinetic-driven flow. The inset shows statistical distribution of v_{eof} measurements; N=68. (C) Current trace as a function of applied electric field. FIG. 6(D) plots v_{eof} as a function of the age of PUMA chips after bonding. Native PUMA exhibited very strong electroosmotic mobility; the EOF moves toward cathode, the same direction as in PDMS, glass, and TPE. This would suggest that the native PUMA surface also exhibited negative charge under the buffer environment used. In borate buffer, v_{eof}, the electroosmotic mobility of PUMA, was 5.5×10⁻⁴ cm²V⁻¹sec⁻¹, quite comparable to that of fused-silica capillary; FIG. 6B inset shows the statistical distribution of electroosmotic mobility measurements. This value is ˜2 times higher than that of thermal-cured polyurethane reported in the literature. FIG. 6B shows how the electrical current stabilized when the anode reservoir was replaced with 20-mM borate buffer. As the EOF drove the 20-mM buffer solution in anode reservoir to displace the 10-mM buffer previously in the channel, the ionic strength increased and led to an increase of electrical current until the entire channel was filled with 20-mM buffer. As the electric field increased from 200 V/cm to 667 V/cm (the maximum output from our power supply), the time to reach a new steady state decreased as expected. Within the range of electric field that we applied, we did not notice any Joule heating. FIG. 6C plots the electrical current measured using 10- and 20-mM borate buffers as a function of the applied electric field. Up to 667 V/cm, these relationships were linear, indicating no alteration in ionic conductivity from Joule heating.

Unlike PDMS or TPE, PUMA surface did not need to be oxidized to achieve high EOF; in addition, the electroosmotic mobility was remarkably stable after manufacturing. FIG. 6′D shows the electroosmotic mobility 641 as measured on different days following manufacturing; to avoid systemic sampling errors associated with sampling from only a single production run, different chips of various ages selected from three production runs were used for each measurement. As shown in FIG. 6′D, the mean (horizontal line 641) was invariant with respect to chip age up to 12 days. However, we did notice an increased frequency of gas bubbles disrupting measurements as chips became older. While we do not know the exact cause of this observation, we had taken great care to rule out common sources of gas bubble by sonicating all solution before use and siphoning out any visible bubbles under microscope inspection. We speculate that perhaps storing PUMA chips in nitrogen or vacuum may help to reduce the incidence of bubble generation.

Unlike PDMS or TPE, PUMA surface did not need to be oxidized to achieve high EOF; in addition, the electroosmotic mobility was remarkably stable after manufacturing.

FIG. 6D shows the electroosmotic mobility as measured on different days following manufacturing; to avoid systemic sampling errors associated with sampling from only a single production run, different chips of various ages selected from three production runs were used for each measurement. As shown in FIG. 6D, the mean (horizontal line) was invariant with respect to chip age up to 12 days. However, we did notice an increased frequency of gas bubbles disrupting measurements as chips became older. While we do not know the exact cause of this observation, we had taken great care to rule out common sources of gas bubble by sonicating all solution before use and siphoning out any visible bubbles under microscope inspection. We speculate that perhaps storing PUMA chips in nitrogen or vacuum may help to reduce the incidence of bubble generation.

In one embodiment, the device for accumulating a biological entity comprises a flow channel defined at least in part within walls of a biocompatible and radiation-absorbing polymer, wherein the device is used for clinical diagnosis.

PUMA is a highly promising material for fabricating microfluidic devices for disposable use in clinical situations. Because the raw material has already been qualified as USP Class VI-compliant, its chemical inertness, working temperature, biocompatibility, and sterilizability have been well characterized and the device fabricated from this material can be expected to meet regulatory approval. This paper reported a finely tuned production process that offered high-fidelity microstructure replication even at high density and high aspect ratio. This production process can be based on either existing PDMS molds fabricated from SU-8-on-Si master or from DRIE-etched Si masters. PUMA offers optical clarity in the visible region and is non-elastomeric. Its surface property is highly stable in comparison with PDMS. Composed mostly of polyurethane, PUMA surface is expected to have similar biofouling resistance as polyurethane. UV-curing process, which takes minutes (<2 min in our procedure, and the UV source may be mounted on a conveyor belt for accurate metering of UV dosage during continuous production) rather than hours as required for thermal curing, is expected to translate to a higher throughput for production, which is needed to bring down the manufacturing costs of disposable microfluidic devices. In addition, as PUMA is a thermoplastic, bonding to form an enclosed microfluidic device is easy and robust: in this instance we simply left the conformally-sealed chips under UV source for an extended period of time. Ultrasonic welding, fast-ramping infrared oven (e.g. often used for re-flowing solder in circuit board repair), or other commercial non-solvent joining approaches may offer additional advantages in quality control. With these characteristics, we anticipate PUMA to be a useful substrate in the fabrication of disposable microfluidic-based diagnostic devices.

Reported above are embodiments of the new UV-curable polyurethane-methacrylate (PUMA) resin that is non-elastomeric and has excellent qualities as a disposable microfluidic substrate, especially for clinical diagnostic applications. This PUMA substrate is transparent optically, resistant to biofouling, compatible with many chemicals encountered in microfluidic applications, curable to a typical thickness (about the thickness of glass slides), bondable to form enclosed devices easily, and capable of generating comparable electroosmotic flow—without surface modification—as a fused-silica capillary. Certified by the supplier as United States Pharmacopeia (USP) Class VI-compliant, this PUMA resin has been tested thoroughly for its chemical inertness, working temperature, biocompatibility, and sterilizability—all qualities necessary for manufacturing medical diagnostic devices.

Also disclosed in this application is a method to form an enclosed microfluidic flow channel, the method comprising:

releasing a formed substrate from a mold;

providing a vacuum to compress the formed substrate against a surface; and

providing an energy to form a seal between the formed substrate and the surface.

In one embodiment, the microfluidic flow channel is configured to flow a biological entity.

In one embodiment, the formed substrate comprises polyurethane-methacrylate (PUMA).

In one embodiment, the formed substrate is formed by exposing a resin to a radiation. In another embodiment, the formed substrate is formed by exposing a resin to a radiation, wherein the radiation has a wavelength between 300-500 nm. In a further embodiment, the formed substrate is formed by exposing a resin to a radiation, wherein the resin contains a urethane, an acrylate, a methacrylate, a silicone, or combinations thereof.

In one embodiment, the formed substrate is released from the mold by pulling at an angle greater than 90 degrees. In another embodiment, the formed substrate is released from the mold by using a vacuum suction.

In some embodiments, the vacuum provided to compress the formed substrate against a surface is contained within a deformable pouch or bag. In one embodiment the deformable pouch or bag encloses the formed substrate and the surface.

In one embodiment, the energy to form a seal between the formed substrate and the surface is a UV radiation. In another embodiment the energy to form a seal between the formed substrate and the surface is a thermal energy or infrared radiation. In a further embodiment the energy to form a seal between the formed substrate and the surface is an oxidizing energy.

The following discussion focuses on the back-end steps—mold-releasing, bonding, and interconnecting to external fluidic delivery—in UV-casting of PUMA resin. During mold-releasing, high-aspect ratio microstructures are prone to shear-induced damage, whereas during bonding, they are prone to compression-related damage. Losses during these two steps must not be convoluted with the yield of UV-casting, which is highly consistent once the UV dosage and the thickness of the resin is properly optimized. We have devoted a great deal of effort to troubleshoot the mold-releasing and bonding steps, and developed techniques to eliminate inconsistencies and inadvertent damages to the replicated microstructures. The result is an increased quality control and improvement in yield. These techniques also can be easily adapted for commercial scale production.

Experimental

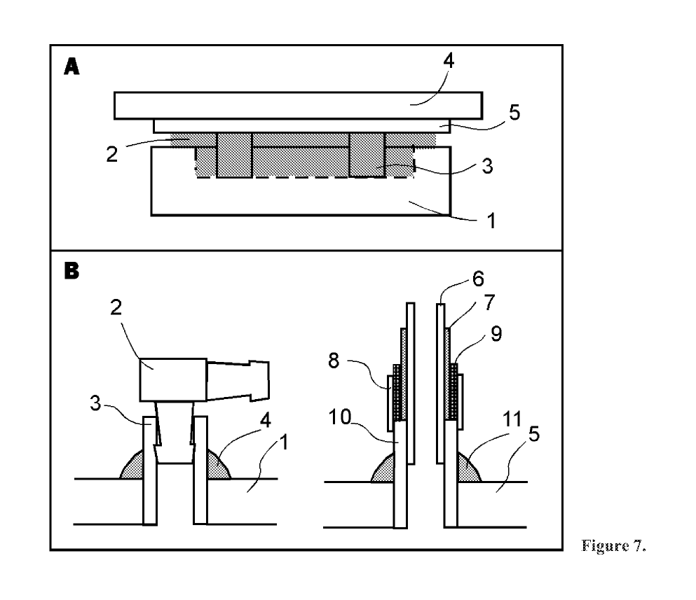

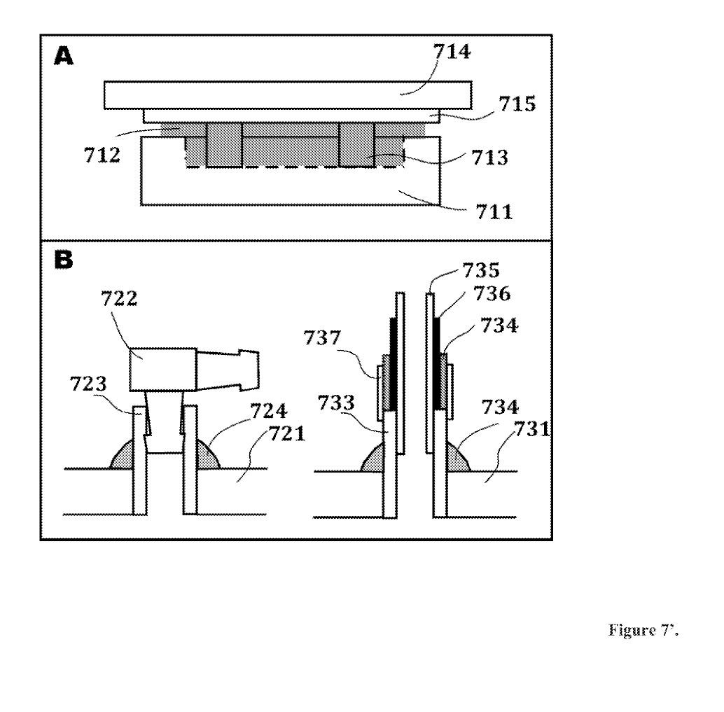

Referring to FIG. 7′, Polydimethylsiloxane (PDMS) molds 711 were prepared according to rapid prototyping procedures described previously except that the molding master was prepared by deep-reactive ion etching (DRIE) of silicon wafer, which was silanized with (tridecafluoro-1,1,2,2-tetrahydrooctyl)trichlorosilane overnight. PUMA resin 712 (Dymax 140-M, Torrington, Conn.) was dispensed to 3-mm thickness onto the PDMS mold 711, then covered with a sheet of cellophane 715 tacked to a clear polypropylene backing 714 (8-mil thick) to prevent oxygen inhibition of the cross-linking reaction (FIG. 7′A).

Polydimethylsiloxane (PDMS) molds were prepared according to rapid prototyping procedures described previously except that the molding master was prepared by deep-reactive ion etching (DRIE) of silicon wafer, which was silanized with (tridecafluoro-1,1,2,2-tetrahydrooctyl)trichlorosilane overnight. PUMA resin (Dymax 140-M, Torrington, Conn.) was dispensed to 3-mm thickness onto the PDMS mold, then covered with a sheet of cellophane tacked to a clear polypropylene backing (8-mil thick) to prevent oxygen inhibition of the cross-linking reaction (FIG. 7A).

Specifically, FIG. 7′A shows a layout showing the molding and curing of PUMA chip. A PDMS mold 711 with a recess of 2-mm deep is filled with PUMA resin 712 and embedded with PTFE posts 713. The top of the resin is covered with a clear polypropylene sheet 714 with an interfacial cellophane (or Aclar) sheet 715, which may be peeled off the resin once cured. 711: PDMS mold; 712: PUMA resin; 713: PTFE posts; 714: clear polypropylene sheet; 715: cellophane (or Aclar). FIG. 7′B is a schematic showing two methods to connect external tubings to the chip. Left: PUMA chip 721 with ⅛-in hole can be connected to a barb connector 722 with a ⅛-in OD polyurethane tubing 723; additional PUMA resin 724 may be dispensed around the tubing to prevent leak. Right: PUMA chip 731 with ⅛-in hole can be connected to a 1/16-in OD PTFE tubing 735. 731: PUMA substrate; 735: 1/16-in OD PTFE tubing; 736: polyolefin heat-shrink; 737: retaining ring; 734: additional adhesive; 733: ⅛-in outer-diameter polyurethane tubing; 734: additional PUMA resin.

Specifically, FIG. 7(A) shows a layout showing the molding and curing of PUMA chip. A PDMS mold 1 with a recess of 2-mm deep is filled with PUMA resin 2 and embedded with PTFE, posts 3. The top of the resin is covered with a clear polypropylene sheet 4 with an interfacial cellophane (or Aclar) sheet 5, which may be peeled off the resin once cured. 1: PDMS mold; 2: PUMA resin; 3: PTFE posts; 4: clear polypropylene sheet; 5: cellophane (or Aclar). FIG. 7(B) is a schematic showing two methods to connect external tubings to the chip. Left: PUMA chip 1 with ⅛-in hole can be connected to a barb connector 2 with a ⅛-in OD polyurethane tubing 3; additional PUMA resin 4 may be dispensed around the tubing to prevent leak. Right: PUMA chip 5 with ⅛-in hole can be connected to a 1/16-in OD PTFE tubing 6. 5: PUMA substrate; 6: 1/16-in OD PTFE tubing; 7: polyolefin heat-shrink; 8: retaining ring; 9: additional adhesive; 10: ⅛-in outer-diameter polyurethane tubing; 11: additional PUMA resin.

Aclar sheets 715 (Honeywell, Morristown, N.J.), which is a polychloro-trifluoroethylene (PCTFE) polymer containing no plasticizer, may be used in lieu of cellophane in critical applications. To form fluidic reservoirs or holes for external connection, PTFE posts 713 (3 mm (D)×3 mm (H)) were embedded in the PUMA resin 712 before curing. The entire assembly was placed in a high-intensity UV source (ADAC Cure Zone 2 UV Flood Light Source, fitted with a 400 W metal halide lamp, providing nominally 80 mW/cm2 at 365 nm) for 80 sec (expose through resin side), followed by an additional 40 sec (expose through mold). Once released from the mold, PUMA substrate was conformally bonded to another PUMA-coated (cured) glass with gentle mechanical pressure. This conformal bond was converted to a permanent bond by placing the PUMA chip under the UV flood source for an additional 10 min.

Aclar sheets (Honeywell, Morristown, N.J.), which is a polychloro-trifluoroethylene (PCTFE) polymer containing no plasticizer, may be used in lieu of cellophane in critical applications. To form fluidic reservoirs or holes for external connection, PTFE posts (3 mm (D)×3 mm (H)) were embedded in the PUMA resin before curing. The entire assembly was placed in a high-intensity UV source (ADAC Cure Zone 2 UV Flood Light Source, fitted with a 400 W metal halide lamp, providing nominally 80 mW/cm2 at 365 nm) for 80 sec (expose through resin side), followed by an additional 40 sec (expose through mold). Once released from the mold, PUMA substrate was conformally bonded to another PUMA-coated (cured) glass with gentle mechanical pressure. This conformal bond was converted to permanent bond by placing the PUMA chip under the UV flood source for an additional 10 min.

Also described in this application is a method to release a formed substrate from a mold by preventing fouling of the mold. The mold is subjected to prolonged washing with a sequence of solvents in presence of acoustic energy.

Between each replication, the PDMS molds were sonicated in isopropanol and water and baked at 75° C. for at least 15 min.

Results and Discussion

Fluidic Interconnect. FIG. 7′B shows two examples of interfacing a PUMA chip for external fluidic delivery. Chips made with these two interfacing methods have routinely withstood up to 40 psi when we applied them to applications involving high volumetric flow rate (1-10 mL/min) or high fluidic resistance. The left side of FIG. 7′B illustrates the use of a 90-degree bend 722 that allows simple attachment of external tubing. The bend 722 was inserted into a thick-wall polyurethane (PU) tubing 723 (⅛-in outer diameter (OD), 1/16-in inner diameter (ID)), which served as a mechanical anchor against shear. The PU tubing 723 was then inserted into a ⅛-in hole (formed either by embedding PTFE posts or laser cutting) in the PUMA substrate 721 and additional adhesive 724 was dispensed around the junction. This design allows quick detachment of the external tubing from the barb connector.

Fluidic Interconnect. FIG. 7B shows two examples of interfacing a PUMA chip for external fluidic delivery. Chips made with these two interfacing methods have routinely withstood up to 40 psi when we applied them to applications involving high volumetric flow rate (1-10 mL/min) or high fluidic resistance. The left side of FIG. 7B illustrates the use of a 90-degree bend that allows simple attachment of external tubing. The bend was inserted into a thick-wall polyurethane (PU) tubing (⅛-in outer diameter (OD), 1/16-in inner diameter (ID)), which served as a mechanical anchor against shear. The PU tubing was then inserted into a ⅛-in hole (formed either by embedding PTFE posts or laser cutting) in the

PUMA substrate and additional adhesive was dispensed around the junction. This design allows quick detachment of the external tubing from the barb connector.

The second design (right side of FIG. 7′B) illustrates interfacing a 1/16-in OD (or of equivalent dimensions as PE100 tubing from Becton Dickinson) PTFE tubing 735 with the PUMA chip 731. We found that conventional polyethylene (PE) tubing (e.g. PE100), which is commonly used for interfacing with PDMS-based microfluidic devices, did not work well with PUMA chips, because (1) PE surfaces are resistant to adhesive bonding, and (2) highly elastic tubings collapse easily when pulled in the longitudinal direction. The best tubing we found was the 1/16-in OD PTFE tubing. Although it is nearly impossible to chemically bond to the PTFE tubing 735, that can be circumvented by covering the external surface with a polyolefin heat-shrink 736. Then the PTFE tubing 735 may be inserted either directly into a 1/16-in diameter hole and secured with additional resin, or into a ⅛-in hole with a supplemental PU tubing 733 (⅛-in OD) as a shear anchor, secured with additional resin 734.

The second design (right side of FIG. 7B) illustrates interfacing a 1/16-in OD (or of equivalent dimensions as PE100 tubing from Becton Dickinson) PTFE tubing with the PUMA chip. We found that conventional polyethylene (PE) tubing (e.g. PE100), which is commonly used for interfacing with PDMS-based microfluidic devices, did not work well with PUMA chips, because (1) PE surfaces are resistant to adhesive bonding, and (2) highly elastic tubings collapse easily when pulled in the longitudinal direction. The best tubing we found was the 1/16-in OD PTFE tubing. Although it is nearly impossible to chemically bond to the PTFE tubing, that can be circumvented by covering the external surface with a polyolefin heat-shrink. Then the PTFE tubing may be inserted either directly into a 1/16-in diameter hole and secured with additional resin, or into a ⅛-in hole with a supplemental PU tubing (⅛-in OD) as a shear anchor, secured with additional resin.

Comparison with PDMS Chips. Cured PUMA resin had a Shore hardness of D 60, which is significantly harder than the elastomeric PDMS (Shore A 50 for Dow Corning's Sylgard 184). For free standing, mechanically fragile features (in particular unsupported tall vertical columns or whiskers), PDMS cannot be used as the material of fabrication because of low shear modulus; the features would simply lean and topple over under gravity.

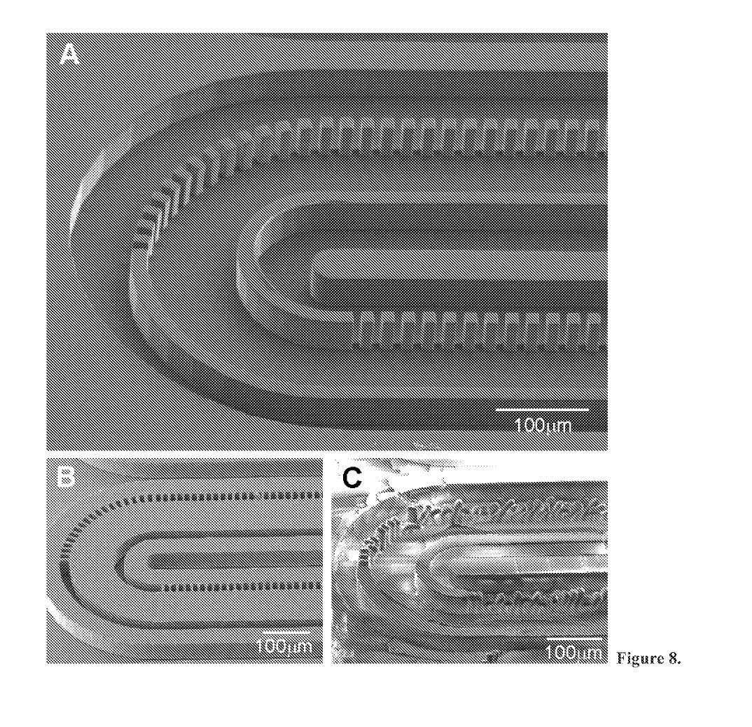

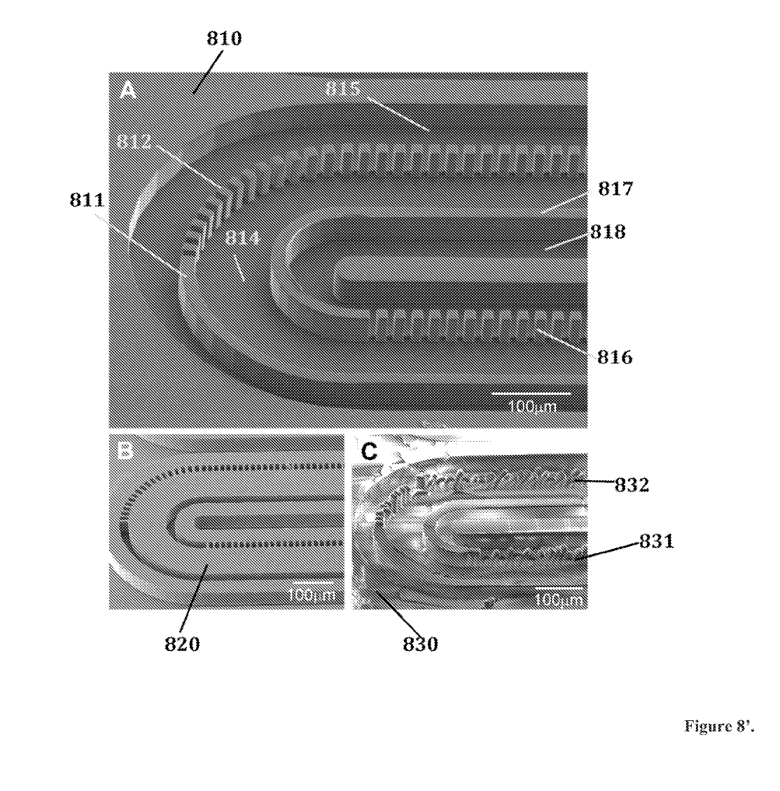

FIG. 8′ shows scanning electron microscopy images of (A) PUMA replica 810 of an array of closely spaced high-aspect ratio columns 812 and 816, (B) DRIE-produced silicon master 820 that is opposite in polarity as (A), and (C) PDMS replica 830 made from the silicon master 820 in (B).

FIG. 8 shows scanning electron microscopy images of (A) PUMA replica of an array of closely spaced high-aspect ratio columns, (B) DRIE-produced silicon master that is opposite in polarity as (A), and (C) PDMS replica made from the silicon master in (B).

FIG. 8′ shows an example of features that can be fabricated in PUMA but not PDMS. FIG. 8′A shows the scanning electron microscopy (SEM) image of a replica 810 in PUMA resin; the test pattern for replication consists of densely spaced vertical columns (812 and 816) alternating with solid walls (811 and 817). The feature height was ˜40 μm and the aspect ratio of the vertical columns (812, 816) was ˜3.5. The bend was incorporated in the design to help troubleshooting if there were directional issues in either the replication or release process. As evident in FIG. 8′A, the columns (812, 816) produced in PUMA had a sharp vertical profile with no evidence of leaning.

FIG. 8 shows an example of features that can be fabricated in PUMA but not PDMS. FIG. 8A shows the scanning electron microscopy (SEM) image of a replica in PUMA resin; the test pattern for replication consists of densely spaced vertical columns alternating with solid walls. The feature height was ˜40 μm and the aspect ratio of the vertical columns was ˜3.5. The bend was incorporated in the design to help troubleshooting if there were directional issues in either the replication or release process. As evident in FIG. 8A, the columns produced in PUMA had a sharp vertical profile with no evidence of leaning.

FIG. 8′B shows a SEM image of a silicon master 820 produced using deep-reactive ion etching (DRIE). This master 820 had an inverse polarity (i.e. relief becomes recess) and was intended for replicating features in PDMS in the same polarity as FIG. 8′A. Whereas SU-8 photoresist on Si wafer is a more common way to produce a master, here the master 820 was produced using DRIE because it was difficult to ensure complete removal of uncured SU-8 resin in deep recesses. The presence of SU-8 in the deep recesses would have contributed to shrinkage of features in the replicated PDMS, which would not be distinguishable from incomplete-filling of PDMS in the recesses. FIG. 8′C shows the PDMS 830 molded from the silicon master 820 in FIG. 8′B. One immediately notices that even though the PDMS columns (831, 832) were of the same height as the long curving walls, which indicates successful replication, they could not support their own weight and thus leaned over. Collapsing or sagging under their own weight is also expected for low-aspect ratio PDMS microchannels.

FIG. 8B shows a SEM image of a silicon master produced using deep-reactive ion etching (DRIE). This master had an inverse polarity (i.e. relief becomes recess) and was intended for replicating features in PDMS in the same polarity as FIG. 8A. Whereas SU-8 photoresist on Si wafer is a more common way to produce a master, here the master was produced using DRIE because it was difficult to ensure complete removal of uncured SU-8 resin in deep recesses. The presence of SU-8 in the deep recesses would have contributed to shrinkage of features in the replicated PDMS, which would not be distinguishable from incomplete-filling of PDMS in the recesses. FIG. 8C shows the PDMS molded from the silicon master in FIG. 8B. One immediately notices that even though the PDMS columns were of the same height as the long curving walls, which indicates successful replication, they could not support their own weight and thus leaned over. Collapsing or sagging under their own weight is also expected for low-aspect ratio PDMS microchannels.

Release Process. We found that for low-aspect ratio (H/W<1) features the cured PUMA resin can be released from the PDMS mold either by (1) peeling the mold slightly away from the cured resin or (2) wedging a scalpel between the resin and the mold to gently lift up the resin. Here, the odds of damaging the relief features during releasing was very low. For high-aspect ratio features, however, especially those that are mechanically fragile due lack of support, the release process plays a pivotal role in the chip yield.

To improve the release process, we tried several surface modification processes on PDMS (e.g. plasma oxidation, silanization with (tridecafluoro-1,1,2,2-tetrahydrooctyl)trichlorosilane, and thin coating of surfactants such as n-dodecyl-beta-D-maltoside (DDM), Gransurf 71, and Gransurf 77). These surface modification techniques did not improve the replication process; in the case of silanization, the surface was simply too hydrophobic for PUMA resin to wet properly. We also tried exploiting differences in thermal expansion (e.g. either quick freeze to −80° C. or heat): thermal treatment caused warping of PDMS in a direction opposite from the cured resin, but the cured resin also globally conformed to the warped PDMS. The result was a warped PUMA resin, rendering the subsequent conformal seal to a planar substrate impossible.

In one embodiment, the method to form an enclosed microfluidic flow channel comprises releasing a formed substrate from a mold, wherein the formed substrate is released from the mold by pulling at an angle greater than 90 degrees. In another embodiment, the method to form an enclosed microfluidic flow channel comprises releasing a formed substrate from a mold, wherein the formed substrate is released from the mold by pulling at an angle greater than 120 degrees, or in other embodiments, at an angle greater than 150 degrees or greater than 180 degrees.

In certain embodiments, the method to form an enclosed microfluidic flow channel comprises releasing a formed substrate from a mold, wherein the formed substrate is released from the mold by using a vacuum suction.

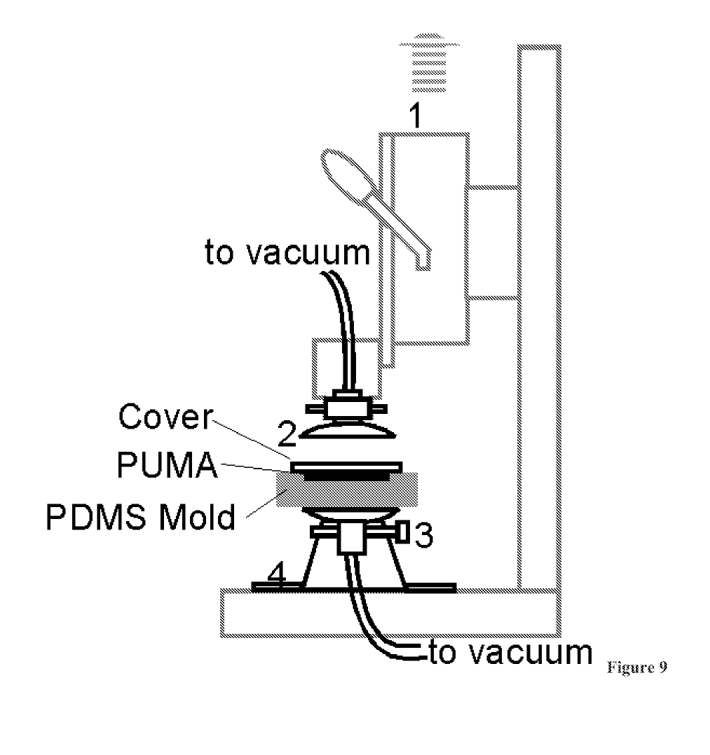

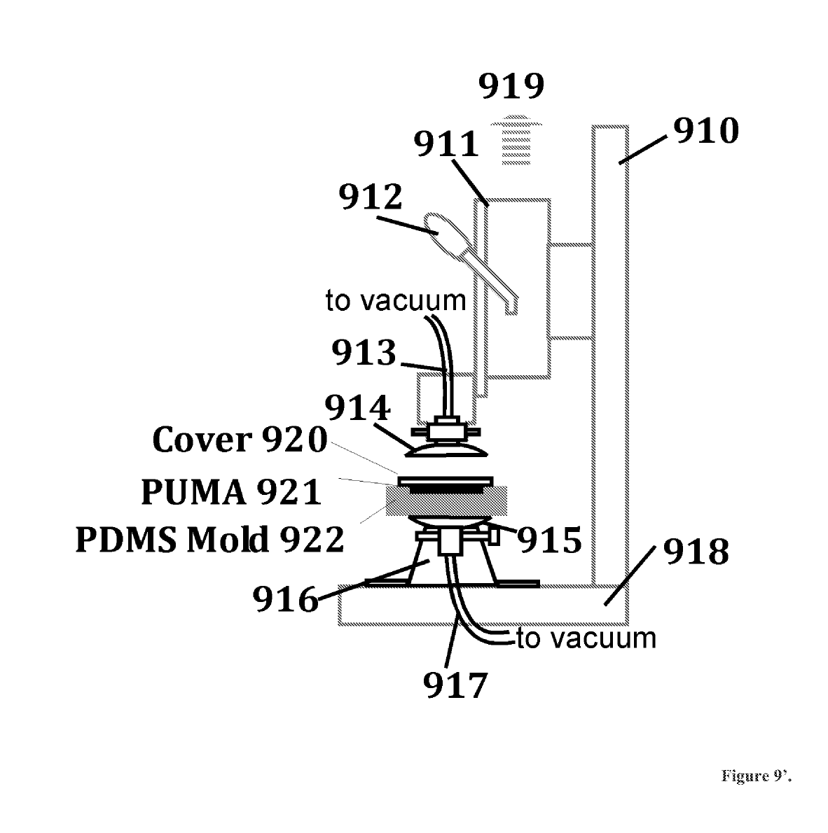

Also described herein, is an apparatus and a method for releasing a formed substrate from a mold by applying opposing vacuum suction forces at an angle greater than 90 degrees. Such an apparatus and a method, as discussed with respect to FIG. 9′, significantly reduces mechanical damages to the replicated microstructures and channels by minimizing inadvertent motion in the shear plane.

Without wishing to be bound to any particular mechanism, it may be that inadvertent mechanical shear must be curbed during the release process, we devised a simple pulling station to separate the cured resin from the PDMS mold. By accurately controlling the direction and the speed of separation, damage to microstructures was greatly minimized.

FIG. 9′ shows a custom-designed release puller 911 for precise release of a PUMA substrate 921 from PDMS mold 922. The puller 911 translates downward when the lever 912 is pulled; upon releasing the lever 912, its spring-loaded action translates upward, ensuring that the PUMA substrate 921 is pulled exactly 180 degrees (direction 919) away from the PDMS mold 922. A 1-in diameter vinyl suction cup 914 was drilled, mounted, and connected to a vacuum pump via a ⅛-inch (inner diameter) Tygon tubing 913. A counter-suction cup 915 was mounted below, also connected to vacuum 917. Metal base 916 was used for securing the counter-suction cup 915 to the Workstation 910.

FIG. 9 shows a custom-designed release puller for precise of a PUMA chip from PDMS mold. The Workstation translates downward when the lever is pulled; upon releasing the lever, its spring-loaded action translates upward, ensuring that the PUMA chip is pulled exactly 180 degrees away from the PDMS mold. Gray outline indicates standard Dremel Workstation components 1. A 1-in diameter vinyl suction cup 2 was drilled, mounted, and connected to a vacuum pump via a ⅛-inch (inner diameter) Tygon tubing. A counter-suction cup 3 was mounted below, also connected to vacuum. Metal base 4 was used for securing the counter-suction cup to the Workstation.

FIG. 9′ shows the schematic of the pulling station 911. It was based on a Dremel Workstation 220-01 assembly 910, which was intended to be a table-top drill press. The Workstation featured a spring-loaded lever 912 that controlled the vertical translation along a shaft; upon releasing the lever 912, the upper mount translated upward until hitting a stop. A 1-in diameter vinyl suction cup 914 was secured to the upper mount for attachment to the PUMA substrate 921, and a second vinyl suction cup 915 for attachment to the PDMS mold 922 was immobilized to a metal base 916. Through holes ( 1/16-in diameter) were drilled at the base of the suction cups 914 and 915 for connecting to a diaphragm vacuum pump.

FIG. 9 shows the schematic of the pulling station. It was based on a Dremel Workstation 220-01 assembly, which was intended to be a table-top drill press. The Workstation featured a spring-loaded lever that controlled the vertical translation along a shaft; upon releasing the lever, the upper mount translated upward until hitting a stop. A 1-in diameter vinyl suction cup was secured to the upper mount for attachment to the PUMA chip, and a second vinyl suction cup for attachment to the PDMS mold was immobilized to a metal base. Through holes ( 1/16-in diameter) were drilled at the base of the suction cups for connecting to a diaphragm vacuum pump.

After UV curing, the PUMA-PDMS assembly (920, 921 and 922) was placed on the base suction cup 915 and the vacuum pump was turned on. The base suction cup 915 held the PDMS mold 922 in place while the upper suction cup 914 was slowly brought down to contact the transparent polypropylene cover 920 on top of the cured resin (formed substrate) 921. The speed should be sufficiently slow such that minimal downward force was exerted on the formed substrate 921. Once the vacuum gauge drops from atmospheric pressure to the ultimate pressure of the pump, indicating that a good vacuum seal was achieved between the upper suction cup 914 and the polypropylene cover 920, the spring-loaded lever 912 was released to pull apart the formed substrate 921 and the mold 922.

After UV curing, the PUMA-PDMS assembly was placed on the base suction cup and the vacuum pump was turned on. The base suction cup held the PDMS mold in place while the upper suction cup was slowly brought down to contact the transparent polypropylene cover on top of the cured resin. The speed should be sufficiently slow such that minimal downward force was exerted on the resin. Once the vacuum gauge drops from atmospheric pressure to the ultimate pressure of the pump, indicating that a good vacuum seal was achieved between the upper suction cup and the polypropylene cover, the spring-loaded lever was released to pull apart the resin and the mold.

We noticed the following in designing the pulling station 911: (1) the upper suction cup 914 and the base suction cup 915 must be properly aligned to distribute forces evenly, and (2) all parts must be securely fastened to avoid inadvertent vibration or motion in the horizontal directions (shear plane of the microstructures). The speed of release (faster the better) also helped to reduce defects.

We noticed the following in designing the pulling station: (1) the upper suction cup and the base suction cup must be properly aligned to distribute forces evenly, and (2) all parts must be securely fastened to avoid inadvertent vibration or motion in the horizontal directions (shear plane of the microstructures). The speed of release (faster the better) also helped to reduce defects.

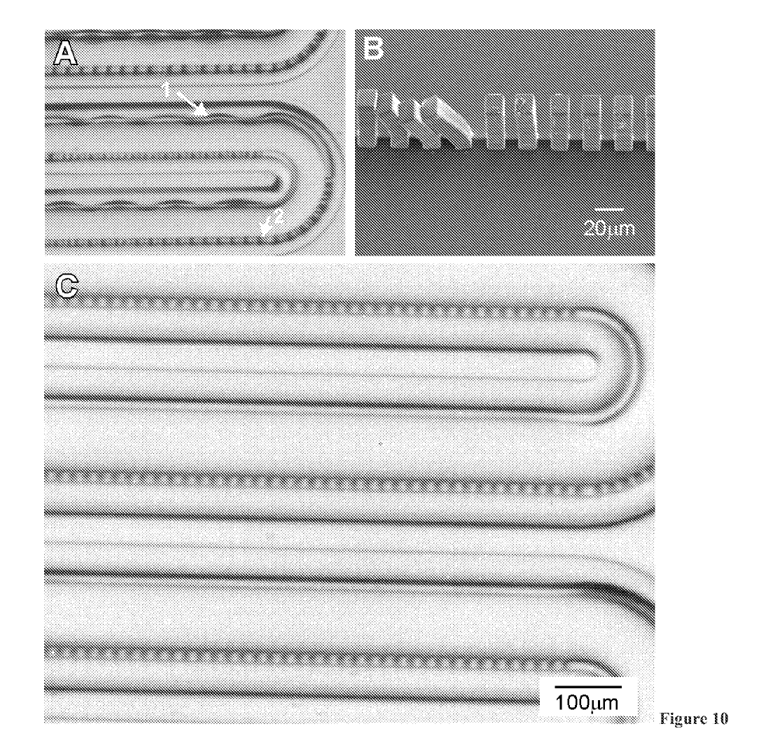

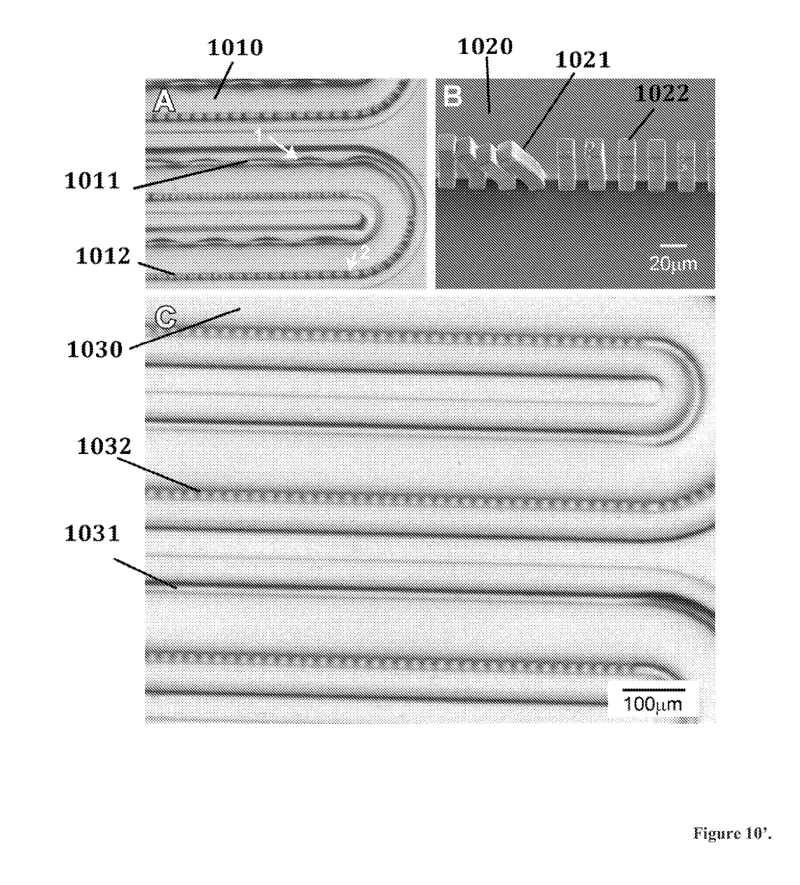

FIG. 10′A shows defects commonly observed under stereoscope for replication of high-aspect ratio structures. Wavy wall 1011 usually results from inadequate cleaning of PDMS mold between each replication run, whereas irregular black spots 1012 amidst regular arrays indicate that the structures were leaning against each other (mechanical damage during releasing PUMA from the PDMS mold). FIG. 10′B is a SEM image 1020 of damaged high-aspect ratio columns 1021; vacuum puller was not used. FIG. 10′C is an optical image of a perfectly released PUMA substrate 1030 using the vacuum puller described earlier.

FIG. 10(A) shows defects commonly observed under stereoscope for replication of high-aspect ratio structures. Wavy wall 1 usually results from inadequate cleaning of PDMS mold between each replication run, whereas irregular black spots 2 amidst regular arrays indicate that the structures were leaning against each other (mechanical damage during releasing PUMA from the PDMS mold). FIG. 10(B) is a SEM image of damaged high-aspect ratio columns; vacuum puller was not used. FIG. 10(C) is an optical image of a perfectly released PUMA chip using the vacuum puller described earlier.

FIG. 10′ shows the improvement in mold-releasing offered by the puller. FIG. 10′A is an image taken under a stereoscope of a PUMA replica 1010 (same pattern as FIG. 8′A) without the assistance of the puller. Two types of defects were evident: (1) the long curvy walls 1011 had a ribbon-like appearance, and (2) the vertical columns 1012 were irregular. The ribbon-appearance of the long curvy wall 1011 came from the wall bending sideways; it is usually due to improper cleaning of the PDMS mold between replication runs, which increases the adhesion between the mold and the resin. Fresh, unused PDMS molds did not exhibit this problem when the curing conditions were strictly followed. Rigorous sonication with isopropanol and water between replications greatly reduced the incidents of wavy walls 1011.

FIG. 10 shows the improvement in mold-releasing offered by the puller. FIG. 10A is an image taken under a stereoscope of a PUMA replica (same pattern as FIG. 8A) without the assistance of the puller. Two types of defects were evident: (1) the long curvy walls had a ribbon-like appearance, and (2) the vertical columns were irregular. The ribbon-appearance of the long curvy wall came from the wall bending sideways; it is usually due to improper cleaning of the PDMS mold between replication runs, which increases the adhesion between the mold and the resin. Fresh, unused PDMS molds did not exhibit this problem when the curing conditions were strictly followed. Rigorous sonication with isopropanol and water between replications greatly reduced the incidents of wavy walls.

FIG. 10′B shows a SEM image of the vertical posts 1021 that would have been deemed “irregular” under stereoscope inspection. The irregularity came from the posts 1021 leaning against each other. Although PUMA is significantly harder than PDMS, at this scale, the features are mechanically fragile. FIG. 10′C shows a stereoscope image of a perfectly released PUMA replica 1030 using the puller. The spacing between the vertical posts was periodic (no irregular dark spots).

FIG. 10B shows a SEM image of the vertical posts that would have been deemed “irregular” under stereoscope inspection. The irregularity came from the posts leaning against each other. Although PUMA is significantly harder than PDMS, at this scale, the features are mechanically fragile. FIG. 10C shows a stereoscope image of a perfectly released PUMA replica using the puller. The spacing between the vertical posts was periodic (no irregular dark spots).

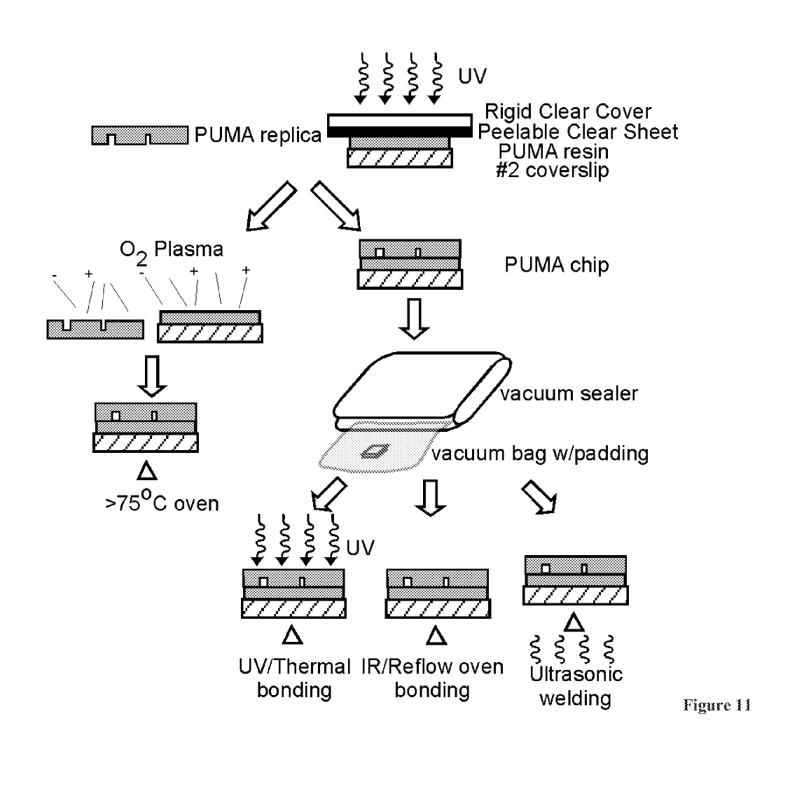

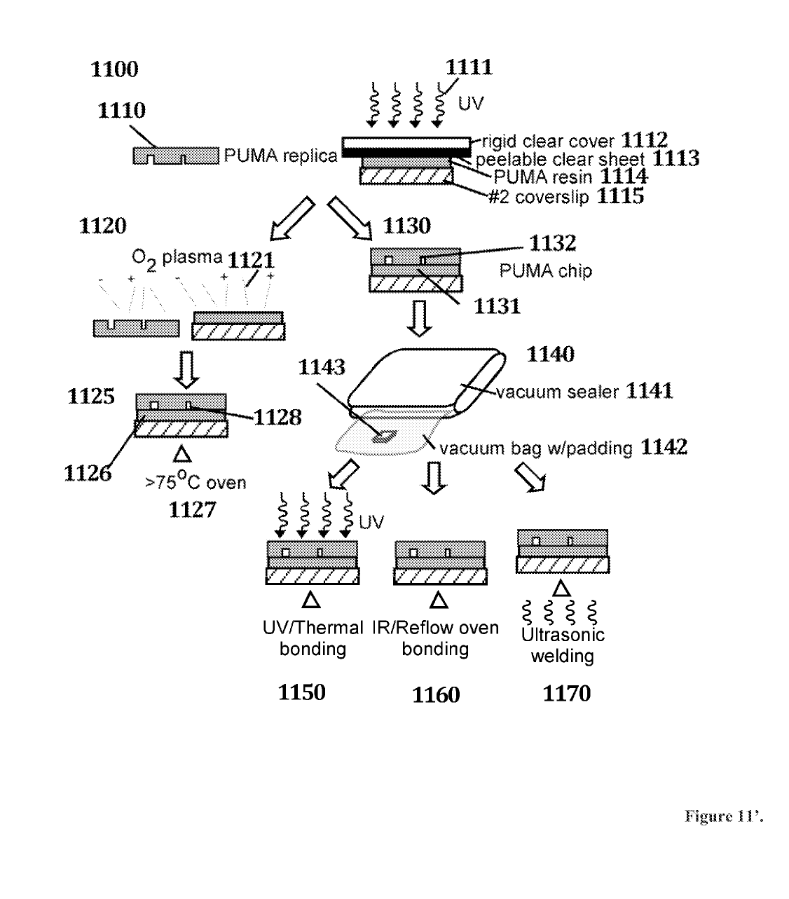

Bonding. FIG. 11′ shows several methods that may be used to form enclosed PUMA microchannels. FIG. 11′. Methods of bonding PUMA chips to form enclosed channels. PUMA chips may be bonded using oxygen plasma 1121 first (step 1120), followed by baking at >75° C. for 2-3 days (step 1125). O₂ plasma 1121 improves the conformal contact between the chip (formed substrate) 1128 and the bottom cover 1126. For high-aspect ratio or delicate structures, we recommend the use of a vacuum sealer 1141 to control the pressure used in conformal seal (step 1140). Once good conformal seal is achieved, a permanent bond may be formed by simply subjecting the chip to extended UV exposure (step 1150), using a programmable infrared oven (step 1160), or ultrasonic welding (step 1170).

Bonding. FIG. 11 shows several methods that may be used to form enclosed PUMA microchannels. FIG. 11. Methods of bonding PUMA chips to form enclosed channels. PUMA chips may be bonded using oxygen plasma first, followed by baking at >75° C. for 23 days. O₂ plasma improves the conformal contact between the chip and the bottom cover. For high-aspect ratio or delicate structures, we recommend the use of a vacuum sealer to control the pressure used in conformal seal. Once good conformal seal is achieved, a permanent bond may be formed by simply subjecting the chip to extended UV exposure, using a programmable infrared oven, or ultrasonic welding.

Since PUMA is a thermoplastic, heat is an effective way to form a permanent bond between the microchannel substrate and the lid. However, to avoid damaging the microstructures, excessive softening or pressure must be avoided during the bonding process.

In certain embodiments, the method to form an enclosed microfluidic flow channel comprises providing a vacuum to compress the formed substrate against a surface. In one embodiment, the vacuum to compress the formed substrate against a surface is contained within a deformable pouch or bag. For example, the pouch or the bag can enclose the formed substrate and the surface.

Also described in this application is an apparatus and a method for providing a vacuum to compress the formed substrate against a surface to form an enclosed flow channel. Such an apparatus and a method, as described with reference to FIG. 11′, provides a vacuum inside a deformable pouch or bag to simultaneously apply a compressive force and remove any trapped air to improve the contact between the formed substrate and the contacting surface.

Referring to FIG. 11′, because of the rigidity of the substrate, conformal seal of PUMA (step 1140) is not as simple as that of PDMS. Care also must be taken to avoid trapped air bubbles. Our preferred method is to place the chip 1143 in a plastic bag 1142, use a vacuum sealer 1141 that is commercially sold as a kitchen appliance to pull a vacuum on the bag, and rely on the collapsing bag to apply pressure evenly on the chip and form the conformal seal. Vacuum bags 1142 often have ridges to reduce trapping of air pockets; these ridges can leave imprints on the PUMA substrate 1143, which can be avoided by lining the vacuum bag 1142 with lint-free cloth.

Because of the rigidity of the substrate, conformal seal of PUMA is not as simple as that of PDMS. Care also must be taken to avoid trapped air bubbles. Our preferred method is to place the chip in a plastic bag, use a vacuum sealer that is commercially sold as a kitchen appliance to pull a vacuum on the bag, and rely on the collapsing bag to apply pressure evenly on the chip and form the conformal seal. Vacuum bags often have ridges to reduce trapping of air pockets; these ridges can leave imprints on the PUMA substrate, which can be avoided by lining the vacuum bag with lint-free cloth.

Following conformal seal (step 1140), the enclosed chips were placed under the UV lamp for 10-15 min (step 1150). The intense UV and heat caused softening of the PUMA substrate and the conformal seal became a permanent bond during the reflow process. The reflow does not usually lead to distortion of microstructures as long as no pressure is applied above the chip while it is still soft. Once the chip cooled, the permanent seal was capable of withstanding high flow rate (>1 ml/min) at high pressure (20-30 psi); we routinely observed that the microscope coverslip (No. 2), which constituted the bottom surface of the chip, fractured before the permanent seal failed. This bonding method 1150 is our method of choice; however, other bonding techniques also may be used, which we describe briefly below.

Following conformal seal, the enclosed chips were placed under the UV lamp for 10-15 min. The intense UV and heat caused softening of the PUMA substrate and the conformal seal became a permanent bond during the reflow process. The reflow does not usually lead to distortion of microstructures as long as no pressure is applied above the chip while it is still soft. Once the chip cooled, the permanent seal was capable of withstanding high flow rate (>1 ml/min) at high pressure (20-30 psi); we routinely observed that the microscope coverslip (No. 2), which constituted the bottom surface of the chip, fractured before the permanent seal failed. This bonding method is our method of choice; however, other bonding techniques also may be used, which we describe briefly below.

In certain embodiments the method to form an enclosed microfluidic flow channel comprises providing an energy to form a seal between the formed substrate and the surface. In some embodiments, the energy is a UV radiation. In other embodiments, the energy is a thermal energy or infrared radiation. In yet another embodiment, the energy is an oxidizing energy, resulting from ion or electron bombardment, exposure to oxygen plasma, or exposure to oxidizing chemicals.

Referring back to FIG. 11′, oxygen plasma (step 1120) may be used to enhance the conformal seal; after 15 minutes of oxygen plasma 1121 the conformal contact was improved. Less air bubbles were trapped and the area of seal increased. However, manual elimination of air bubbles was still required because the sealing area usually was nowhere near the 100% as typically witnessed between PDMS and glass. The permanent bond was formed when the enclosed chip (1128 and 1126) was placed in a 75° C. oven for two days; however, using this procedure, the frequency of seal failure during experiments was higher than with the chips produced using the first bonding method described above.

Oxygen plasma may be used to enhance the conformal seal; after 15 minutes of oxygen plasma the conformal contact was improved. Less air bubbles were trapped and the area of seal increased. However, manual elimination of air bubbles was still required because the sealing area usually was nowhere near the 100% as typically witnessed between PDMS and glass. The permanent bond was formed when the enclosed chip was placed in a 75° C. oven for two days; however, using this procedure, the frequency of seal failure during experiments was higher than with the chips produced using the first bonding method described above.

Alternate solventless-bonding methods that bear more resemblance to commercial production of thermoplastics may also be used. For example, programmable infrared oven (step 1160), which provides fast ramping of temperature and is frequently used for reflowing solder in circuit-board fabrication, should provide a more reliable temperature control than the UV lamp. Ultrasonic welding (step 1170), which is a common technique for joining thermoplastics, may also be used provided the operating condition is properly optimized to reduce microstructure damage from local melting.

Alternate solventless-bonding methods that bear more resemblance to commercial production of thermoplastics may also be used. For example, programmable infrared oven, which provides fast ramping of temperature and is frequently used for reflowing solder in circuit-board fabrication, should provide a more reliable temperature control than the UV lamp. Ultrasonic welding, which is a common technique for joining thermoplastics, may also be used provided the operating condition is properly optimized to reduce microstructure damage from local melting.

In one embodiment, the device for accumulating a biological entity comprises a flow channel defined at least in part within walls of a biocompatible and radiation-absorbing polymer, wherein the biological entity is a cancer cell. In another embodiment, the biological entity is a rare cell (e.g., a cell of low abundance). A cell may be considered as rare if its concentration is 1) less than 10% of the total cell population in a fluid, 2) less than 1% of the total cell population in a fluid, or 3) less than 1 million cells per milliliter of a fluid.

In certain embodiments the device comprising a flow channel defined at least in part within walls of a biocompatible and radiation-absorbing polymer may be used to accumulate a biological entity. The flow channel may be further used for electrophoresis, electrochromatography, chromatography, high pressure liquid chromatography (HPLC), filtration, surface selective capture (including selective antibody-protein capture, DNA hybridization, enzyme linked immunosorbent assay (ELISA)) DNA amplification, polymerase chain reaction (PCR), Southern blot analysis, cell culturing, proliferation assay, or other assay, or combinations thereof. In a further embodiment, the device may be used for clinical diagnosis.

In certain embodiments the device comprising a flow channel defined at least in part within walls of polyurethane-methacrylate (PUMA), may be used to accumulate a biological entity. The flow channel may be used for electrophoresis, electrochromatography, chromatography, high pressure liquid chromatography (HPLC), filtration, surface selective capture (including selective antibody-protein capture, DNA hybridization, enzyme linked immunosorbent assay (ELISA)) DNA amplification, polymerase chain reaction (PCR), Southern blot analysis, cell culturing, proliferation assay, or other assay, or combinations thereof. In a further embodiment, the device may be used for clinical diagnosis.

In certain embodiments the device for accumulating a biological entity comprises a flow channel defined at least in part within walls of a biocompatible and radiation-absorbing polymer, wherein at least one of the walls defining the flow channel is coated with an antibody.

Examples of antibodies for surface selective capture include but are not limited to the pan-cytokeratin antibody A45B/B3, AE1/AE3, or CAM5.2 (pan-cytokeratin antibodies that recognize Cytokeratin 8 (CK8), Cytokeratin 18 (CK18), or Cytokeratin 19 (CK19) and ones against: breast cancer antigen NY-BR-1 (also known as B726P, ANKRD30A, Ankyrin repeat domain 30A); B305D isoform A or C (B305D-A ro B305D-C; also known as antigen B305D); Hermes antigen (also known as Antigen CD44, PGP1); E-cadherin (also known as Uvomorulin, Cadherin-1, CDH1); Carcino-embryonic antigen (CEA; also known as CEACAM5 or Carcino-embryonic antigen-related cell adhesion molecule 5); β-Human chorionic gonadotophin (β-HCG; also known as CGS, Chronic gonadotrophin, (β polypeptide); Cathepsin-D (also known as CTSD); Neuropeptide Y receptor Y3 (also known as NPY3R; Lipopolysaccharide-associated protein3, LAP3, Fusion; Chemokine (CXC motif, receptor 4); CXCR4); Oncogene ERBB1 (also known as c-erbB-1, Epidermal growth factor receptor, EGFR); Her-2 Neu (also known as c-erbB-2 or ERBB2); GABA receptor A, pi (π) polypeptide (also known as GABARAP, GABA-A receptor, pi (π) polypeptide (GABA A(π), γ-Aminobutyric acid type A receptor pi (π) subunit), or GABRP); ppGalNac-T(6) (also known as β-1-4-N-acetyl-galactosaminyl-transferase 6, GalNActransferase 6, GalNAcT6, UDP-N-acetyl-d-galactosamine:polypeptide N-acetylgalactosaminyltransferase 6, or GALNT6); CK7 (also known as Cytokeratin 7, Sarcolectin, SCL, Keratin 7, or KRT7); CK8 (also known as Cytokeratin 8, Keratin 8, or KRT8); CK18 (also known as Cytokeratin 18, Keratin 18, or KRT18); CK19 (also known as Cytokeratin 19, Keratin 19, or KRT19); CK20 (also known as Cytokeratin 20, Keratin 20, or KRT20); Mage (also known as Melanoma antigen family A subtytpes or MAGE-A subtypes); Mage3 (also known as Melanoma antigen family A 3, or MAGA3); Hepatocyte growth factor receptor (also known as HGFR, Renal cell carninoma papillary 2, RCCP2, Protooncogene met, or MET); Mucin-1 (also known as MUC1, Carcinoma Antigen 15.3, (CA15.3), Carcinoma Antigen 27.29 (CA 27.29); CD227 antigen, Episialin, Epithelial Membrane Antigen (EMA), Polymorphic Epithelial Mucin (PEM), Peanut-reactive urinary mucin (PUM), Tumor-associated glycoprotein 12 (TAG12)); Gross Cystic Disease Fluid Protein (also known as GCDFP-15, Prolactin-induced protein, PIP); Urokinase receptor (also known as uPR, CD87 antigen, Plasminogen activator receptor urokinase-type, PLAUR); PTHrP (parathyrold hormone-related proteins; also known as PTHLH); BS 106 (also known as B511S, small breast epithelial mucin, or SBEM); Prostatein-like Lipophilin B (LPB, LPHB; also known as Antigen BU101, Secretoglobin family 1-D member 2, SCGB1-D2); Mammaglobin 2 (MGB2; also known as Mammaglobin B, MGBB, Lacryglobin (LGB) Lipophilin C (LPC, LPHC), Secretoglobin family 2A member 1, or SCGB2A1); Mammaglobin (MGB; also known as Mammaglobin 1, MGB1, Mammaglobin A, MGBA, Secretoglobin family 2A member 2, or SCGB2A2); Mammary serine protease inhibitor (Maspin, also known as Serine (or cystein) proteinase inhibitor clade B (ovalbumin) member 5, or SERPINB5); Prostate epithelium-specific Ets transcription factor (PDEF; also known as Sterile alpha motif pointed domain-containing ets transcription factor, or SPDEF); Tumor-associated calcium signal transducer 1 (also known as Colorectal carcinoma antigen CO17-1A, Epithelial Glycoprotein 2 (EGP2), Epithelial glycoprotein 40 kDa (EGP40), Epithelial Cell Adhesion Molecule (EpCAM), Epithelial-specific antigen (ESA), Gastrointestinal tumor-associated antigen 733-2 (GA733-2), KS1/4 antigen, Membrane component of chromosome 4 surface marker 1 (M4S 1), MK-1 antigen, MIC 18 antigen, TROP-1 antigen, or TACSTD1); Telomerase reverse transcriptase (also known as Telomerase catalytic subunit, or TERT); Trefoil Factor 1 (also known as Breast Cancer Estrogen-Inducible Sequence, BCEI, Gastrointestinal Trefoil Protein, GTF, pS2 protein, or TFF1); folate; or Trefoil Factor 3 (also known as Intestinal Trefoil Factor, ITF, p1.B; or TFF3).

In one embodiment, the device for accumulating a biological entity comprises a flow channel defined at least in part within walls of a biocompatible and radiation-absorbing polymer, wherein the biological entity is a cell, organelle, bacteria, virus, protein, antibody, DNA, or a bioconjugated particle.

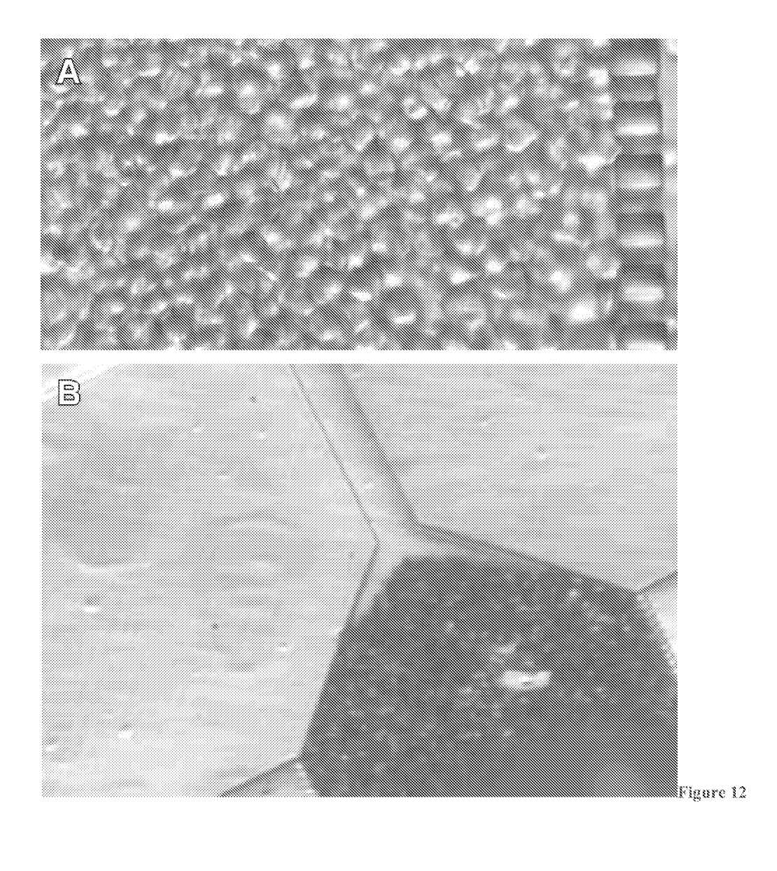

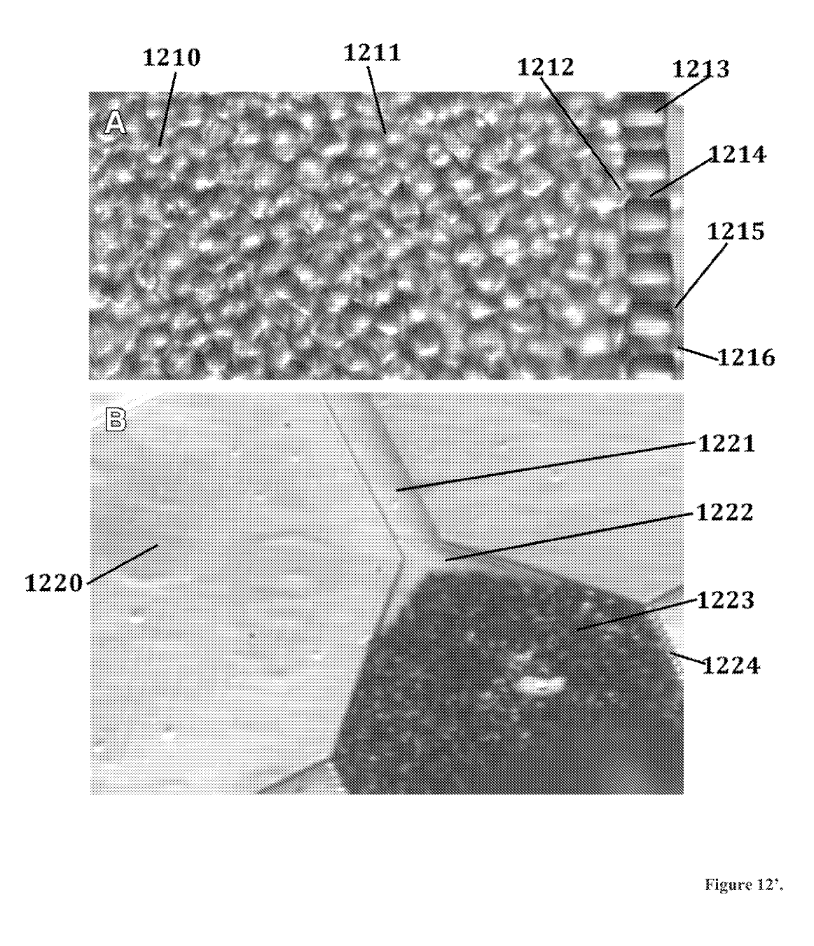

Application. One motivation that drove our development of the PUMA chip was the need to fabricate a dense array of high-aspect ratio slits for applications in microfiltration. FIG. 12′ shows microscope images in which a dense packing of cells 1211 (FIG. 12′A) and beads 1223 (FIG. 12′B) were retained, trapped, and accumulated by an array of vertical columns or fins 1213 produced in PUMA.

Application. One motivation that drove our development of the PUMA chip was the need to fabricate a dense array of high-aspect ratio slits for applications in microfiltration. FIG. 12 shows microscope images in which a dense packing of cells (FIG. 12A) and beads (FIG. 12B) were retained and trapped by an array of vertical columns or fins produced in PUMA.

In particular, FIG. 12′A shows retention and accumulation of MCF-7 cancer cells 1211 by high-aspect ratio slits 1214 (right side of image) fabricated in PUMA resin. Nominal flow rate was 0.3 ml/min; cells were fixed in 4% paraformaldehyde for 15 min. FIG. 12′B shows retention and accumulation of 15 μm-diameter beads 1223 by high-aspect ratio slits 1224 made from PUMA resin. The same microfluidic design was used for (A) and (B), where a filtration barrier 1213 comprising the high-aspect ratio slits 1214 was placed at the exit 1222 of the microchannel 1221.

In particular, FIG. 12(A) shows retention of MCF-7 cancer cells by high-aspect ratio slits (right side of image) fabricated in PUMA resin. Nominal flow rate was 0.3 ml/min; cells were fixed in 4% paraformaldehyde for 15 min. FIG. 12(B) shows retention of 15 μm-diameter beads by high-aspect ratio slits made from PUMA resin. The same microfluidic design was used for (A) and (B), where a filtration barrier comprising the high-aspect ratio slits was placed at the exit of the microchannel.

In one aspect, “accumulation” does not require the depletion of another similar species. Accumulation refers to an increase in the absolute number of a species. Enrichment by depleting a second species which results in an increase in the ratio with respect to the second species is not the same as accumulation. For example, if in the beginning there are 10 species A and 10 species B (1:1 ratio), and at the end there are 10 species A and 2 species B (5:1 ratio), that is enrichment but not accumulation, since the absolute number of species A has not increased.

In both experiments, the same microfluidic design was used, where the distance between the columns 1213 was 8 μm and the height of the column 1213 was 40 μm. In FIG. 12′A, a dilute solution of fixed cultured cancer cells 1211 (MCF-7 cells fixed in 4% paraformaldehyde for 15 min) was used and flowed through the chip at 0.3 ml/min. Such microfluidic filter may serve to complement existing grid-based manual haemacytometer for clinical diagnostic use, because the ability to concentrate cells into a small area allows for a more accurate and rapid enumeration of cells, especially when the cells are present at a highly diluted concentration. In FIG. 12′B, a solution of 15 μm-diameter beads 1223 was used.

This capability to pack beads in a microchannel may also find broad use, such as in affinity purification (e.g. where the beads were conjugated with antibodies) or in size-exclusion chromatography. For all such microfiltration-based applications, it is imperative to be able to fabricate the filtration elements with high yield, because failure to replicate a single fin will result in the failure of the entire chip. This paper shows that PUMA possesses the material property for fabricating such demanding microfluidic systems, provided that care is taken and that the described microfabrication procedure is followed.

In both experiments, the same microfluidic design was used, where the distance between the columns was 8 μm and the height of the column was 40 μm. In FIG. 12A, a dilute solution of fixed cultured cancer cells (MCF-7 cells fixed in 4% paraformaldehyde for 15 min) was used and flowed through the chip at 0.3 ml/min. Such microfluidic filter may serve to complement existing grid-based manual haemacytometer for clinical diagnostic use, because the ability to concentrate cells into a small area allows for a more accurate and rapid enumeration of cells, especially when the cells are present at a highly diluted concentration. In FIG. 12B, a solution of 15 μm-diameter beads was used. This capability to pack beads in a microchannel may also find broad use, such as in affinity purification (e.g. where the beads were conjugated with antibodies) or in size-exclusion chromatography. For all such microfiltration-based applications, it is imperative to be able to fabricate the filtration elements with high yield, because failure to replicate a single fin will result in the failure of the entire chip. This paper shows that PUMA possesses the material property for fabricating such demanding microfluidic systems, provided that care is taken and that the described microfabrication procedure is followed.

In conclusion, PUMA is a highly promising substrate for commercial production of microfluidic chips for clinical diagnostic applications. Because PUMA is a non-elastomeric substrate, extra care must be taken to avoid damaging high-aspect-ratio microstructures during mold-releasing or during bonding to form an enclosed microfluidic device. The UV-curing process of PUMA resin is highly robust; however, improper release or bonding can significantly reduce the chip yield. We showed that by using a release puller that minimizes motion in the shear plane of the microstructures, high-aspect ratio microstructures can be perfectly replicated even in a high-density array, such as those used in our microfiltration chip. To avoid excessive compressive forces during conformal seal, a vacuum sealer should be used to remove the air between the PUMA replica and the bottom surface of the chip, while utilizing the collapsing vacuum bag to exert a gentle yet even compressive force. Once conformal seal has been established, various bonding strategies can be used to convert this conformal seal to a permanent bond, including the use of a UV lamp to further cure and heat the chip, a process that offers high yield and a strong bond. The ability of PUMA to replicate high-aspect-ratio microstructure should find use for a wide range of analytical applications, and we believe PUMA will complement existing substrates in the production of disposable microfluidic devices, especially those that will be used in a clinical setting.

Attached hereto as Exhibits A and B are copies of two articles that are incorporated by reference in their entireties herein for all purposes. Attached hereto as Exhibit C is a product sheet for an example of a material for use in accordance with embodiments of the present invention.

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the claims, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above and so the claims should not be limited to the devices or methods described herein. While some processes are described in a given order, alternative embodiments may perform methods having steps in a different order, and some processes may be deleted, moved, added, subdivided, combined, and/or modified. Accordingly, each of these methods may be implemented in a variety of different ways. Also, while some methods are at times shown as being performed in series, these methods may instead be performed in parallel, or may be performed at different times. The headings provided herein are for convenience only and do not interpret the scope or meaning of the claims.

The terminology used in the description is intended to be interpreted in its broadest reasonable manner, even though it is being used in conjunction with a detailed description of identified embodiments.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. When the claims use the word “or” in reference to a list of two or more items, that word covers all of the following interpretations of the word: any of the items in the list, all of the items in the list, and any combination of the items in the list.

Any patents, applications and other references, including any that may be listed in accompanying filing papers, are incorporated herein by reference. Aspects of the described technology can be modified, if necessary, to employ the systems, functions, and concepts of the various references described above to provide yet further embodiments.

These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the claims to the specific embodiments disclosed in the specification, unless the above Detailed Description section explicitly defines such terms. Accordingly, the actual scope of the claims encompasses not only the disclosed embodiments, but also all equivalents.

We claim:

1. A device for accumulating a biological entity, the device comprising a flow channel defined at least in part within walls of a biocompatible and radiation-absorbing polymer.

2. The device of claim 1 wherein the polymer comprises polyurethane-methacrylate (PUMA).

3. The device of claim 1 wherein the polymer absorbs radiation at wavelengths between 300-500 nm.

4. The device of claim 1 wherein the polymer is biocompatible according to an injection test, an intracutaneous test, an implantation test, or combinations thereof.

5. The device of claim 1 wherein the polymer comprises a urethane, an acrylate, a methacrylate, a silicone, or combinations thereof.

6. The device of claim 1 wherein the polymer is a thermoplastic.

7. The device of claim 1 wherein the polymer is nonelastomeric.

8. The device of claim 1 wherein the walls are resistant against an oil, an acid, and/or a base.

9. The device of claim 1 wherein the biological entity is a cell, organelle, bacteria, virus, protein, antibody, DNA, or a bioconjugated particle.

10. The device of claim 9 wherein the cell is of low abundance in a sample.

11. The device of claim 9 wherein the cell is a cancer cell.

12. The device of claim 1 wherein at least one of the walls defining the flow channel is coated with an antibody.

13. The device of claim 1 wherein the walls do not autofluoresce.

14. The device of claim 1 wherein the walls are formed by crosslinking a medical grade adhesive.

15. The use of a device comprising a flow channel defined at least in part within walls of polyurethane-methacrylate (PUMA) to accumulate a biological entity.

16. The use of claim 15 wherein the flow channel is used for electrophoresis, electrochromatography, high pressure liquid chromatography, filtration, surface selective capture, DNA amplification, polymerase chain reaction, Southern blot analysis, cell culturing, cell proliferation assay, or combinations thereof.

17. The use of claim 15 wherein the device is used for clinical diagnosis.

18. A method to form an enclosed microfluidic flow channel, the method comprising
releasing a formed substrate from a mold;
providing a vacuum to compress the formed substrate against a surface; and
providing an energy to form a seal between the formed substrate and the surface.

19. The method of claim 18 wherein the microfluidic flow channel is configured to flow a biological entity.

20. The method of claim 18 wherein the formed substrate comprises polyurethane-methacrylate (PUMA).

21. The method of claim 18 wherein the formed substrate is formed by exposing a resin to radiation.

22. The method of claim 21 wherein the radiation has a wavelength between 300-500 nm.

23. The method of claim 21 wherein the resin contains a urethane, an acrylate, a methacrylate, a silicone, or combinations thereof.

24. The method of claim 18 wherein the formed substrate is released from the mold by pulling at an angle greater than 90 degrees.

25. The method of claim 18 wherein releasing the formed substrate from the mold comprises releasing using a vacuum suction.

26. The method of claim 18 wherein providing a vacuum comprises providing the vacuum within a deformable pouch.

27. The method of claim 18 wherein providing the energy comprises providing the energy selected from oxidizing energy, UV radiation, thermal energy, or infrared radiation.
